(19) United States
(12) Patent Application Publication
(10) Pub. No.: US 2020/0056224 A1
(43) Pub. Date: Feb. 20, 2020

(54) BARCODED TRANSPOSASES TO INCREASE EFFICIENCY OF HIGH-ACCURACY GENETIC SEQUENCING

(71) Applicant: Fred Hutchinson Cancer Research Center, Seattle, WA (US)

(72) Inventor: Jason Bielas, Seattle, WA (US)

(73) Assignee: Fred Hutchinson Cancer Research Center, Seattle, WA (US)

(21) Appl. No.: 16/606,640

(22) PCT Filed: Apr. 18, 2018
(86) PCT No.: PCT/US18/28204

§ 371 Date: Oct. 18, 2019

Related U.S. Application Data
(60) Provisional application No. 62/486,836, filed on Apr. 18, 2017.

(51) Int. Cl. C12Q 1/6806; C12N 9/22; C12N 15/10

(52) U.S. Cl. CPC C12Q 1/6806; C12N 15/1096; C12N 9/22

(57) ABSTRACT

Transposase-based barcoding of DNA for improved efficiency, high-accuracy sequencing is described. Transposases including transposable barcodes can be used to fragment and barcode target DNA in a single step. The transposase-based system to prepare samples for high-accuracy sequencing lead to shorter processing times and fewer processing steps, greatly enhancing the efficiency and accuracy of genetic sequencing

### CROSS-REFERENCE TO RELATED APPLICATION

This application claims priority to U.S. Provisional Patent Application No. 62/486,836 filed on Apr. 18, 2017, which is incorporated herein by reference in its entirety as if fully set forth herein.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 17-080-WO-PCT_ST25.txt. The text file is 8.2 KB, was created on Apr. 18, 2018, and is being submitted electronically via EFS-Web.

### FIELD OF THE DISCLOSURE

The current disclosure provides transposase-based barcoding systems to prepare DNA samples for high accuracy genetic sequencing. The transposase-based barcoding systems increase the efficiency of genetic sequencing procedures and allow differentiation between (i) errors that occur during genetic sequencing; and (ii) rare sequence variants.

### BACKGROUND OF THE DISCLOSURE

The ability to sequence the genetic code has vastly improved our understanding of biology and has ushered in a new era in research and therapeutic medicine. The genomes of all living organisms are made of deoxyribonucleic acid (DNA) or ribonucleic acid (RNA). DNA or RNA is composed of strings of nucleotides represented by letters as follows: DNA is composed of the nucleotides: (i) A (adenine), (ii) C (cytosine), (iii) G (guanine) and (iv) T (thymine) while RNA is composed of (i) A, (ii) C, (iii) G, and (iv) U (uracil). Genetic sequencing involves determining the order of the nucleotides in a genome or portion of a genome. For a human, the number of nucleotides in a genome is 3 billion, often expressed as 3 billion base (nucleotide) pairs, as DNA occurs as two strands of nucleotides intertwined in a helical configuration. The ability to sequence genomes is very powerful, as mutations, or variations in the genetic sequence of each person's genome, may underlie diseases such as cancer. Sequence information can help guide prediction and treatment of diseases. Outside of the disease setting, genetic sequencing can also be useful in endeavors such as evaluating organism populations in environments and assessing how different organisms relate to one another and evolve.

First generation sequencing used a method called chain termination. However, this method was labor intensive and not amenable to scale-up to sequence multiple genomes or very large genomes. Next generation sequencing (NGS), also referred to as massively parallel or deep sequencing, allows greater speed and accuracy in sequencing, with concomitant reduction in manpower and cost.

Mutations in a genetic sequence can include substitutions (substituting one nucleotide for another in the genetic sequence), deletions (deletion of one or more nucleotides from the genetic sequence), and insertions (inserting one or more nucleotides into the genetic sequence). Although NGS can be utilized to detect rare sequence mutations (e.g., naturally occurring mutations) in a DNA sample, the error rate of NGS can make it difficult to distinguish between true rare sequence mutations and artefactual mutations that occur due to preparation of a DNA sample for sequencing or during the sequencing itself. For example, NGS can exhibit an error rate of 5 substitution errors per 10,000 nucleotides to 1 substitution error per 100 nucleotides. Gregory et al. (2016) Nucleic Acids Research 44(3): e22. Given that some mutation frequencies in cancerous tissues can be 1 mutation per 1 million nucleotides, rare sequence mutations cannot be detected in the background of errors created by NGS DNA sample preparation and sequencing.

Numerous steps and extended processing time can be required to prepare and sequence DNA samples, thus contributing to errors that confound detection of true rare mutations. For example, currently preparing samples for NGS can require (i) fragmenting the DNA into more manageable lengths for sequencing; (ii) ligating A tails, or stretches of adenine nucleotides, to the ends of the fragmented DNA for attaching tag sequences that enable sequencing; (iii) attaching the tags to the fragmented DNA; and (iv) making numerous copies of, or amplifying, each tagged fragmented DNA by a process called polymerase chain reaction (PCR).

Among other potential sequences, tag sequences can include barcodes and adapters. A barcode can be a random stretch of nucleotides that serves as a unique tag to identify a DNA molecule that is sequenced. A barcode is useful because each barcode allows one to track every sequence generated back to an original DNA fragment that was sequenced. Adapters are composed of short nucleotide sequences that can allow immobilization of a DNA fragment to a solid surface for the sequencing and/or provide regions on the DNA fragment from which the sequencing process can start. In particular, asymmetrical adapters allow one to track every sequence generated back to one strand of a double-stranded DNA fragment that was sequenced. The output of a sequencing is sequence reads, which are strings of nucleotide letters for every strand of every double-stranded DNA fragment that is sequenced. Sequence reads with the same barcode can then be grouped together and differences among the sequence reads in a given barcode family can be readily detected. Differences in sequences at a given nucleotide position can represent true mutations if they occur in the majority of the sequence reads, while non-relevant mutations arising from errors due to experimental processes such as PCR and sequencing can occur in a minority of the reads. Therefore, a consensus sequence can be generated for each DNA fragment that represents a true, accurate sequence for that DNA fragment. A consensus sequence can show nucleotide positions that are constant (i.e., always represented by the same nucleotide in all samples) versus positions that include different nucleotides depending on the presence of a naturally occurring mutation.

Methods have been developed to increase the efficiency of sample processing for certain applications of NGS. For example, fragmentation of DNA was typically carried out using a sonicator, a nebulizer, or enzymes that cut up DNA. However, these processes could lead to significant loss of the DNA sample and required additional steps to select the DNA fragments. Therefore, an alternative method to fragment DNA for sequencing was developed that took advantage of the action of proteins called transposases. A transposase is an enzyme that binds to the end of a DNA segment called a transposon and catalyzes the movement of the transposon from one part of the genome to another part of the genome. The transposition results in the excision of the whole transposon from the first region and insertion of the transposon into the second region.

A method called in vitro transposition using transposases has been developed to cut and add tags to nucleic acids. It was discovered that engineering a double-stranded DNA to only include sequences found at the ends of a transposon (and not the whole transposon) still allowed a transposase to recognize and bind the transposon end sequences in a complex. This complex of transposase and transposon end sequences was able to bind to a target site in DNA (either a specific or non-specific target sequence), make a cut at the target site, and insert the transposon end sequence into the target site by ligating, or joining, the end of the cut target DNA to the transposon end. The result was DNA cut wherever a transposase/transposon end sequences complex bound, with transposon end sequences ligated to the cut ends of the DNA. Thus, the in vitro transposition method offers a more streamlined route to preparation of DNA for sequencing, but this process in and of itself does not lead to a DNA sample that is ready for high accuracy genetic sequencing.

Use of NGS for high accuracy genetic sequencing requires more complicated techniques, but these techniques still require multiple steps and/or specialized equipment. For example, a protocol to accurately sequence ancient DNA includes two purification steps and a step to remove damaged bases to reduce error. Briggs and Heyn (2012) Methods Mol Biol 840: 143-154. As another example, a microfluidic device, where extremely small volumes of fluids can be manipulated, was designed to consolidate sample preparation steps that included isolation of the genomic DNA, fragmentation and tagging with transposases, and DNA purification. Kim et al. (2017) Nat Commun 8: 13919. However, in some instances larger fluid samples are needed. Thus, methods to increase efficiency of preparing samples for high accuracy genetic sequencing are still needed.

### SUMMARY OF THE DISCLOSURE

The current disclosure provides systems and methods to increase efficiency of preparing DNA samples for high accuracy genetic sequencing. The systems and methods use transposases with transposable barcodes and asymmetrical adapters. Use of a transposase with transposable barcodes reduces the sample processing steps required to perform next generation DNA sequencing (NGS) because the multiple steps of fragmenting DNA, preparing the ends of the DNA for attachment of tags, and attachment of tags are collapsed into one or two steps rather than three steps generally performed before the amplification step. The reduction in processing steps saves time and can also reduce the number of errors that occur due to sample processing. The systems and methods of the present disclosure allow the preparation of sequencing-ready, barcoded, fragmented DNA having asymmetrical adapters at the ends of the fragmented DNA. The presence of barcodes allows tracking of sequence reads to an original sequenced nucleic acid fragment, while the presence of asymmetrical adapters allow tracking of sequence reads to a particular strand of an original sequenced nucleic acid fragment. Thus, using a transposase-based system with transposable barcodes and asymmetrical adapters reduces the steps for sample preparation, and the concomitant incorporation of barcodes and asymmetrical adapters enable the generation of consensus sequences for high accuracy sequencing.

In particular embodiments, the transposase includes a E54K/L372P Tn5 transposase. In particular embodiments, the transposable barcodes are transposable due to the presence of transposon end sequences. In particular embodiments, the transposon ends are mosaic ends, or hyperactive versions of transposon ends. In particular embodiments, the transposable barcodes can further include a spacer region. In particular embodiments, sample fragmentation, attachment of barcodes, tail ligation, and ligation of asymmetrical adapters can be achieved in a single processing step.

### BRIEF DESCRIPTION OF THE FIGURES

FIG. 1 shows a schematic of transposase-based fragmentation and barcoding for high accuracy genetic sequencing.

FIG. 2 provides exemplary sequences disclosed herein.

FIG. 3 shows HCT116 genomic DNA tagmented with Tn5 transposase-bound barcoded adapters. Size distribution of tagmented genomic DNA fragments decreases with decreasing input mass. DNA input from left to right: 20 ng, 30 ng, 40 ng, 50 ng.

FIGS. 4A and 4B show characterization of a human error-corrected sequencing library. (FIG. 4A) The distribution of barcode families in the library. (FIG. 4B) Whole genome mutation spectrum with (black) and without (gray) error correction. Read mapping quality was Q30. For error correction, a minimum of two read-sequence clusters was required for each strand of the original input DNA molecule.

### DETAILED DESCRIPTION

The ability to sequence the genetic code has vastly improved our understanding of biology and has ushered in a new era in research and therapeutic medicine. The genomes of all living organisms are made of deoxyribonucleic acid (DNA) or ribonucleic acid (RNA). DNA or RNA is composed of strings of nucleotides represented by letters as follows: DNA is composed of (i) A (adenine), (ii) C (cytosine), (iii) G (guanine) and (iv) T (thymine) while RNA is composed of (i) A, (ii) C, (iii) G, and (iv) U (uracil). Genetic sequencing involves determining the order of the nucleotides in a genome or portion of a genome. For a human, the number of nucleotides in a genome is 3 billion, often expressed as 3 billion base (nucleotide) pairs, as DNA occurs as two strands of nucleotides intertwined in a helical configuration. The ability to sequence genomes is very powerful, as mutations, or variations in the genetic sequence of each person's genome, may underlie diseases such as cancer. Sequence information can help guide prediction and treatment of diseases. Outside of the disease setting, genetic sequencing can also be useful in endeavors such as evaluating organism populations in environments and assessing how different organisms relate to one another and evolve.

First generation sequencing used a method called chain termination. However, this method was labor intensive and not amenable to scale-up to sequence multiple genomes or very large genomes. Next generation sequencing (NGS), also referred to as massively parallel or deep sequencing, allows greater speed and accuracy in sequencing, with concomitant reduction in manpower and cost.

Mutations in a genetic sequence can include substitutions (substituting one nucleotide for another in the genetic sequence), deletions (deletion of one or more nucleotides from the genetic sequence), and insertions (inserting one or more nucleotides into the genetic sequence). Although NGS can be utilized to detect rare sequence mutations (e.g., naturally occurring mutations) in a DNA sample, the error rate of NGS can make it difficult to distinguish between true rare sequence mutations and artefactual mutations that occur due to preparation of a DNA sample for sequencing or during the sequencing itself. For example, NGS can exhibit an error rate of 5 substitution errors per 10,000 nucleotides to 1 substitution error per 100 nucleotides. Gregory et al. (2016) Nucleic Acids Research 44(3): e22. Given that some mutation frequencies in cancerous tissues can be 1 mutation per 1 million nucleotides, rare sequence mutations cannot be detected in the background of errors created by NGS DNA sample preparation and sequencing.

Numerous steps and extended processing time can be required to prepare and sequence DNA samples, thus contributing to errors that confound detection of true rare mutations. For example, currently preparing samples for NGS can require (i) fragmenting the DNA into more manageable lengths for sequencing; (ii) ligating A tails, or stretches of adenine nucleotides, to the ends of the fragmented DNA for attaching tag sequences that enable sequencing; (iii) attaching the tags to the fragmented DNA; and (iv) making numerous copies of, or amplifying, each tagged fragmented DNA by a process called polymerase chain reaction (PCR).

Among other potential sequences, tag sequences can include barcodes and adapters. A barcode can be a random stretch of nucleotides that serves as a unique tag to identify a DNA molecule that is sequenced. A barcode is useful because each barcode allows one to track every sequence generated back to an original DNA fragment that was sequenced. Adapters are composed of short nucleotide sequences that can allow immobilization of a DNA fragment to a solid surface for the sequencing and/or provide regions on the DNA fragment from which the sequencing process can start. In particular, asymmetrical adapters allow one to track every sequence generated back to one strand of a double-stranded DNA fragment that was sequenced. The output of a sequencing is sequence reads, which are strings of nucleotide letters for every strand of every double-stranded DNA fragment that is sequenced. Sequence reads with the same barcode can then be grouped together and differences among the sequence reads in a given barcode family can be readily detected. Differences in sequences at a given nucleotide position can represent true mutations if they occur in the majority of the sequence reads, while non-relevant mutations arising from errors due to experimental processes such as PCR and sequencing can occur in a minority of the reads. Therefore, a consensus sequence can be generated for each DNA fragment that represents a true, accurate sequence for that DNA fragment. A consensus sequence can show nucleotide positions that are constant (i.e., always represented by the same nucleotide in all samples) versus positions that include different nucleotides depending on the presence of a naturally occurring mutation.

Methods have been developed to increase the efficiency of sample processing for certain applications of NGS. For example, fragmentation of DNA was typically carried out using a sonicator, a nebulizer, or enzymes that cut up DNA. However, these processes could lead to significant loss of the DNA sample and required additional steps to select the DNA fragments. Therefore, an alternative method to fragment DNA for sequencing was developed that took advantage of the action of proteins called transposases. A transposase is an enzyme that binds to the end of a DNA segment called a transposon and catalyzes the movement of the transposon from one part of the genome to another part of the genome. The transposition results in the excision of the whole transposon from the first region and insertion of the transposon into the second region.

A method called in vitro transposition using transposases has been developed to cut and add tags to nucleic acids. It was discovered that engineering a double-stranded DNA to only include sequences found at the ends of a transposon (and not the whole transposon) still allowed a transposase to recognize and bind the transposon end sequences in a complex. This complex of transposase and transposon end sequences was able to bind to a target site in DNA (either a specific or non-specific target sequence), make a cut at the target site, and insert the transposon end sequence into the target site by ligating, or joining, the end of the cut target DNA to the transposon end. The result was DNA cut wherever a transposase/transposon end sequences complex bound, with transposon end sequences ligated to the cut ends of the DNA. Thus, the in vitro transposition method offers a more streamlined route to preparation of DNA for sequencing, but this process in and of itself does not lead to a DNA sample that is ready for high accuracy genetic sequencing.

Use of NGS for high accuracy genetic sequencing requires more complicated techniques, but these techniques still require multiple steps and/or specialized equipment. For example, a protocol to accurately sequence ancient DNA includes two purification steps and a step to remove damaged bases to reduce error. Briggs and Heyn (2012) Methods Mol Biol 840: 143-154. As another example, a microfluidic device, where extremely small volumes of fluids can be manipulated, was designed to consolidate sample preparation steps that included isolation of the genomic DNA, fragmentation and tagging with transposases, and DNA purification. Kim et al. (2017) Nat Commun 8: 13919. However, in some instances larger fluid samples are needed. Thus, methods to increase efficiency of preparing samples for high accuracy genetic sequencing are still needed.

The current disclosure provides systems and methods to increase efficiency of preparing DNA samples for high accuracy genetic sequencing. The systems and methods use transposases with transposable barcodes and asymmetrical adapters. Use of a transposase with transposable barcodes reduces the sample processing steps required to perform next generation DNA sequencing (NGS) because the multiple steps of fragmenting DNA, preparing the ends of the DNA for attachment of tags, and attachment of tags are collapsed into one or two steps rather than three steps generally performed before the amplification step. The reduction in processing steps saves time and can also reduce the number of errors that occur due to sample processing. The systems and methods of the present disclosure allow the preparation of sequencing-ready, barcoded, fragmented DNA having asymmetrical adapters at the ends of the fragmented DNA. The presence of barcodes allows tracking of sequence reads to an original sequenced nucleic acid fragment, while the presence of asymmetrical adapters allow tracking of sequence reads to a particular strand of an original sequenced nucleic acid fragment. Thus, using a transposase-based system with transposable barcodes and asymmetrical adapters reduces the steps for sample preparation, and the concomitant incorporation of barcodes and asymmetrical adapters enable the generation of consensus sequences for high accuracy sequencing.

In particular embodiments, the transposase includes a E54K/L372P Tn5 transposase. In particular embodiments, the transposable barcodes are transposable due to the presence of transposon end sequences. In particular embodiments, the transposon ends are mosaic ends, or hyperactive versions of transposon ends. In particular embodiments, the transposable barcodes can further include a spacer region. In particular embodiments, sample fragmentation, attachment of barcodes, tail ligation, and ligation of asymmetrical adapters can be achieved in a single processing step. In particular embodiments, the transposase-based systems with transposable barcodes and asymmetrical adapters increase the efficiency of genetic sequencing procedures and allow differentiation between (i) errors that occur during preparation of nucleic acid molecules for sequencing or during genetic sequencing; and (ii) rare sequence variants.

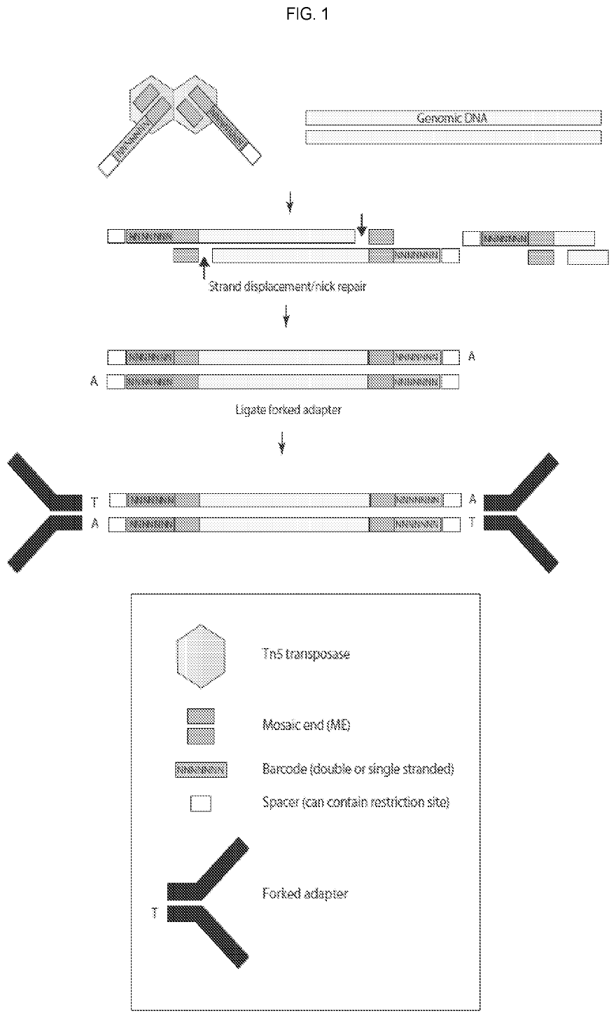

Referring to FIG. 1, in particular embodiments, a transposase can be used for fragmenting and adding barcodes to DNA samples during preparation for sequencing. In particular embodiments, the transposases include unique barcodes and mosaic ends.

The following aspects of the disclosure are now described in additional detail: (i) Transposases; (ii) Transposons and Transposon Ends; (iii) Transposable Barcodes and Spacers; (iv) A- and T-tails; (v) Asymmetrical Adapters; (vi) Transposase-Based Systems; (vii) Methods of Preparing a Nucleic Acid Sample for High Accuracy Sequencing; (viii) Error Correction; and (ix) Kits.

(i) Transposases. A transposase of the disclosure can be any protein having transposase activity in vitro. In particular embodiments, a transposase is an enzyme that is capable of forming a functional complex with a nucleic acid including a transposon end and a unique barcode, and as part of the functional complex, binding to and cutting (fragmenting) a double-stranded target DNA, and joining the transposon end and unique barcode at the end of the double-stranded target DNA. In particular embodiments, the fragmentation and tagging of a target DNA occurs when the target DNA is incubated with one or more transposase/nucleic acid complexes in an in vitro transposition reaction. A transposase can be a naturally occurring transposase or a recombinant transposase. In particular embodiments, the transposase can be in cell lysates of cells in which the transposase is produced. In particular embodiments, the transposase can be isolated or purified from its natural environment (i.e., cell nucleus or cytosol). In particular embodiments, the transposase can be recombinantly produced, and isolated or purified from the recombinant host environment (i.e., cell nucleus or cytosol) prior to inclusion in transposase-based systems of the present disclosure.

In particular embodiments, the transposase is a DDE motif transposase such as a prokaryotic transposase from ISs, Tn3, Tn5, Tn7, or Tn10; a bacteriophage transposase from phage Mu; or a eukaryotic “cut and paste” transposase. U.S. Pat. Nos. 6,593,113; 9,644,199; Yuan and Wessler (2011) Proc Natl Acad Sci USA 108(19):7884-7889. In particular embodiments, the transposase includes a retroviral transposase, such as HIV. Rice and Baker (2001) Nat Struct Biol. 8: 302-307.

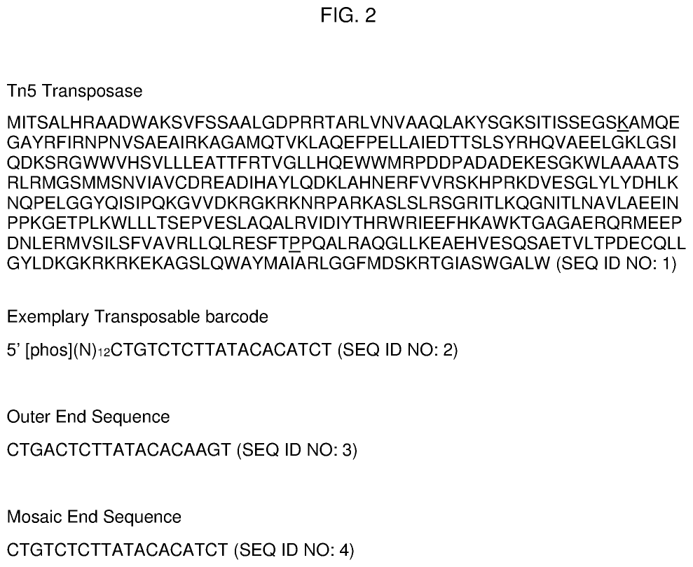

In particular embodiments, the transposase is a member of the IS50 family of transposases, such as Tn5 transposase or variants of Tn5 transposase. Tn5 transposase is derived from the Tn5 transposon, a bacterial transposon that can encode antibiotic resistance genes. The activity of Tn5 transposase can be increased with the point mutations E54K and/or L372P. In particular embodiments, the transposase is a E54K/L372P mutant of Tn5 transposase, which has increased transposase activity. An exemplary E54K/L372P Tn5 transposase is SEQ ID NO: 1 (FIG. 2). Other mutations to increase the activity of Tn5 transposase are disclosed in U.S. Pat. Nos. 5,965,443; 6,406,896; 7,608,434; and Reznikoff (2003) Molecular Microbiology 47(5): 1199-1206. In particular embodiments, the Tn5 transposase is a mutant transposase (Tn5-059) with a lowered GC insertion bias. Kia et al. (2017) BMC Biotechnology 17: 6.

In particular embodiments, a transposase is associated, by way of chemical bonding, to a nucleic acid including a unique barcode and a transposon end. In particular embodiments, a transposase binds a nucleic acid including a unique barcode and a transposon end. In particular embodiments, the nucleic acid includes a double-stranded transposon end. In particular embodiments, the nucleic acid includes a single-stranded unique barcode. In particular embodiments, the nucleic acid includes a double-stranded unique barcode. In particular embodiments, the nucleic acid includes a spacer.

A complex of two transposases can represent a form similar to a synaptic complex. Higher order complexes are also possible, for example, complexes including four transposases, eight transposases, or a mixture of different numbers of sizes of complexes. In a transposase-based system including more than two transposases, not all transposases need be bound by nucleic acids including unique barcodes and transposon ends, as long as there are at least two transposases, each having a bound nucleic acid including a unique barcode and a transposon end.

In particular embodiments, one or more of the transposases in a transposase-based system of the disclosure can be partially or wholly inactive via modification of their amino acid sequences, and a mixture of active and partially or wholly inactive transposase molecules can modulate the distance between active subunits, consequently allowing the modulation of the average size of DNA fragments produced by a transposase-based system.

In particular embodiments, complexes including transposases recognizing different sequences in target DNA can be used, for example, a complex including a transposase that recognizes target DNA sequences having high GC content (and conversely, low AT content) and another transposase that recognizes target DNA sequences having lower GC content (and conversely, high AT content). In particular embodiments, GC or AT content can be expressed as a percentage value, for example, % GC content=(G+C)/(A+T+G+C)*100. In particular embodiments, a high GC content can include 55% to 95% GC, or 60% to 90% GC, or 65% to 85%, or 70% to 80%, or 75% to 80%. In particular embodiments, lower GC content can include 5% to 45%, or 10% to 40%, or 15% to 35%, or 20% to 30%, or 25% to 30%. Mixing of transposases recognizing target DNA sequence differing in GC or AT content allows for tailoring of fragmentation patterns of the target DNA.

In particular embodiments, a transposase can include a tag for purification or immobilization on a support. In particular embodiments, tagging systems that can be used include: avidin or streptavidin/biotin; nano-tag/streptavidin; antibody/antigen such as anti-Myc antibody/Myc tag or anti-FLAG™ antibody/FLAG™ tag (available from e.g., Thermo Fisher Scientific, Waltham, Mass.); enzyme/substrate such as glutathione transferase/reduced glutathione; poly-histidine/nickel-based resin; aptamers/specific target molecules; and Si-tag/silica particles. In particular embodiments, a transposase can be fused to intein and chitin-binding domain. Picelli et al. (2014) Genome Research 24: 2033-2040.

(ii) Transposons and Transposon Ends. Examples of transposons from which transposon ends can be obtained or derived include Tn5, Mu, sleeping beauty (e.g., derived from the genome of salmonid fish); piggyBac (e.g., derived from lepidopteran cells and/or Myotis lucifugus); mariner (e.g., derived from Drosophila); frog prince (e.g., derived from Rana pipiens); Tol2 (e.g., derived from medaka fish); TcBuster (e.g., derived from the red flour beetle Tribolium castaneum) and spinON.

In particular embodiments, transposon end includes a double-stranded DNA that includes only the nucleotide sequences (the “transposon end sequences”) that are necessary to form a complex with the transposase that is functional in an in vitro transposition reaction. A transposon end forms a complex with a transposase that recognizes and binds to the transposon end, and the complex is capable of inserting or transposing the transposon end into target DNA with which it is incubated in an in vitro transposition reaction. A transposon end exhibits two complementary sequences including a “transferred transposon end sequence” or “transferred strand” and a “non-transferred transposon end sequence,” or “non-transferred strand”. Examples of transposon end sequences include the Tn5 outer end and the mosaic end. The Tn5 outer end is a sequence that is encoded by wild-type Tn5 and can include the sequence CTGACTCTTATACACAAGT (SEQ ID NO: 3; FIG. 2). In particular embodiments, the mosaic end is an artificial mutant of the Tn5 outer end and can include the sequence CTGTCTCTTATACACATCT (SEQ ID NO: 4; FIG. 2). In particular embodiments, a transposon end becomes ligated to the 5′ end of a target DNA fragment. In particular embodiments, transposon end sequences are suitably designed such that each transposase can bind a transposon end. In particular embodiments, one or more sequences that a transposase can bind to can be used to design a transposon end sequence. In particular embodiments, a transposon end sequence includes a single recognition sequence for a particular transposase. In particular embodiments, a transposon end sequence includes two or more recognition sequences for a same transposase. In particular embodiments, the efficiency of transposase fragmentation can be assessed separately for several recognition sequences, and recognition sequences with the same efficiency are included in transposon end sequences for use together in a given nucleic acid including a unique barcode and a transposon end, or in separate nucleic acids including unique barcodes and transposon ends. In particular embodiments, a transposon end sequence can include a native transposon end sequence or an engineered sequence that differs in nucleotide sequence from the native sequence. In particular embodiments, a single type of natural or engineered transposon end sequence can be used, or simultaneously two or more types of natural or engineered transposon end sequences can be used. In particular embodiments, a transposon end includes a mosaic end. In particular embodiments, a transposon end sequence is derived from mariner/Tc1 Mos1 transposon and can include 5′-AAACGACATTTCATACTTGTACACCTGA-3′ (SEQ ID NO: 5) and 5′-TTTGCTGTAAAGTATGAACATGTGG-3′ (SEQ ID NO: 6). Morris et al. (2016) eLife 5:e15537. In particular embodiments, a transposon end sequence is derived from Musca domestica Hermes transposon and can include: 5′-CTTGTTGTTGTTCTCTG-3′ (SEQ ID NO: 7) and 5′-GAACAACAACAAGAGAC-3′ (SEQ ID NO: 8); 5′-CTTGTTGAAGTTCTCTG-3′ (SEQ ID NO: 9) and 5′-GAACAACTTCAAGAGAC-3′ (SEQ ID NO: 10). Hickman et al. (2014) Cell 158: 353-367; US 2015/0284768.

(iii) Transposable Barcodes and Spacers. Barcodes refer to nucleic acid sequences that can be utilized to identify the origin of a sample. In particular embodiments, barcodes are DNA sequences. In the context of the present disclosure, a barcode allows a sequence in a complex mixture of sequences to be connected back to an original nucleic acid molecule that was sequenced. In particular embodiments, barcodes can be used to computationally deconvolute the sequencing data and map all sequence reads to single molecules to distinguish library preparation and/or sequencing errors from real mutations. Forked adapters can be incorporated in fragmented DNA in a transposase-based system of the present disclosure and used in combination with barcodes to map all sequence reads to a specific strand of a given fragmented DNA molecule.

In particular embodiments, these barcodes can be designed to be unique. In particular embodiments, DNA barcodes can include standardized short sequences of DNA (400-800 bp) characterized, in theory, for all species on the planet. Kress and Erickson, Proc. Natl. Acad. Sci. USA, 105(8): 2761-2762; Savolainen et al., Trans R Soc London Ser B. 2005; 360:1805-1811. An error correction barcode can be a unique nucleotide sequence used to identify sequencing reads that originate from the same DNA template fragment. In particular embodiments, the error correction barcode is 5-20 nucleotides long. In particular embodiments, the error correction barcode is 12 nucleotides long. In particular embodiments the error correction barcode is a series of random nucleotides. In particular embodiments, barcodes can be designed based on Hamming codes. Hamming codes are a family of binary linear error-correcting codes that can be used to identify substitution errors. In particular embodiments, using barcodes based on Hamming codes can allow error detection and correction of barcodes. Bystrykh (2012) PLoS ONE 7(5): e36852.

In particular embodiments, a barcode is a transposable barcode because it has a transposon end. In particular embodiments, a transposable barcode includes a single-stranded barcode and a double-stranded transposon end at the 3′ end of the single-stranded barcode. In particular embodiments, a transposable barcode includes a single-stranded barcode, a double-stranded transposon end at the 3′ end of the single-stranded barcode, and a single-stranded spacer at the 5′ end of the single-stranded barcode. In particular embodiments, a transposable barcode includes a double-stranded barcode and a double-stranded transposon end at the 3′ end of the double-stranded barcode. In particular embodiments, a transposable barcode includes a double-stranded barcode, a double-stranded transposon end at the 3′ end of the double-stranded barcode, and a double-stranded spacer at the 5′ end of the double-stranded barcode. In particular embodiments, a transposable barcode includes a double-stranded barcode, a double-stranded transposon end at the 3′ end of the double-stranded barcode, and a double-stranded region of non-complementarity at the 5′ end of the double-stranded barcode that can serve as priming sites to add adapters on by PCR. In particular embodiments, a transposable barcode includes a double-stranded barcode, a double-stranded transposon end at the 3′ end of the double-stranded barcode, and an asymmetrical adapter (see below) at the 5′ end of the double-stranded barcode.

In particular embodiments, a transposable high diversity barcode library is a plurality of at least 1,000; at least 10,000; at least 100,000; at least 1,000,000; at least 100,000,000; or at least 1,000,000,000 unique (i.e., non-identical) transposable barcodes, each unique sequence including a transposon end at the 3′ end and an error correction barcode of 5-20 random nucleotides 5′ to the transposon end. In particular embodiments, the transposable barcodes include the sequence 5′-[phos](N)₁₂CTGTCTCTTATACACATCT (SEQ ID NO: 2; FIG. 2), wherein N can be any nucleotide. In particular embodiments, the non-transferred strand of a transposable barcode is blocked by modifications at the 3′ end to prevent the strand from acting as a primer. Examples of modifications at the 3′ end of a nucleic acid to prevent polymerization from that end include use of dideoxycytidine, a phosphate group, a phosphate ester group, an inverted 3′-3′ linkage, and a C3 spacer (3 hydrocarbon) CPG. In particular embodiments, the transposable barcodes include the sequence 5′-[phos]NNNNNNNNNNAGATGTGTATAAGAGACAG (SEQ ID NO: 11).

In particular embodiments, the transposable barcode includes a spacer. In particular embodiments, spacer sequences can include any sequence of nucleotides. In particular embodiments, spacer sequences can include AATT, TTGC, CCGC, TATGG, ATCCT, GGAATT, GCATAG, GCGGATC, GCGGATCT, and AGTGCCAG. In particular embodiments, the spacer and the transposon end are present at opposite ends of the transposable barcode. In particular embodiments, the spacer is 3-15 nucleotides. In particular embodiments the spacer is 4-6 nucleotides. In particular embodiments, the spacer does not include dinucleotide repeats. In particular embodiments, a spacer can protect a barcode from exonucleases and other types of damage to DNA ends. In particular embodiments, a spacer can provide more clearly resolved sequencing results for the barcode sequence. In particular embodiments, the spacer includes a restriction site.

Because in particular embodiments the systems and methods of the present disclosure include a transposase and a transposable barcode, target DNA can be fragmented and tagged with barcodes in one step, thus reducing the amount of steps required for preparing samples for NGS. In particular embodiments, a DNA fragment includes a portion or piece or segment of a target DNA that is cleaved from or released or broken from a longer DNA molecule such that it is no longer attached to the parent molecule. The process of generating DNA fragments from the target DNA is referred to as “fragmenting” the target DNA. In some embodiments, the plurality of fragmented DNA molecules have a size range of 100-3000 bp, or 100-250 bp, or 250-500 bp, or 500-750 bp, or 750-1000 bp, or 1000-1250 bp, or 1250-1500 bp, or 1500-1750 bp, or 1750-2000 bp, or 2000-2250 bp, or 2250-2500 bp, or 2500-2750 bp, or 2750-3000 bp. In particular embodiments, a process of fragmenting DNA and tagging the fragmented DNA with one or more tags or barcodes is called tagmentation.

(iv) A- and T-Tails. In particular embodiments, A-tails or T-tails are added to the barcoded DNA fragments to facilitate ligation to asymmetrical adapters. A-tailing is the addition of non-templated adenosine overhangs to the 3′ end of a double-stranded DNA molecule. A-tailed DNA can be useful for ligation to DNA with a T-overhang at the 3′ end. T-tails are non-templated thymine overhangs added to the 3′ end of a double-stranded DNA molecule. T-tails can be useful for ligation to A-tailed DNA. Enzymes that can add 3′ A-tails or T-tails to double stranded DNA include Taq polymerase, terminal transferase, poly(A) polymerase, Klenow and Klenow fragment.

(v) Asymmetrical Adapters. Transposase-barcoded fragments can be ligated to asymmetrical adapters that provide non-identical primer binding sites for amplification of distinct PCR products derived from each complementary strand. Asymmetrical adapters can refer to adapters that are partially single-stranded, due to the presence of one or more regions of non-complementarity between the sense strand and the antisense strand, and partially double-stranded or capable of forming a duplex structure, due to the presence of one or more regions of complementarity between the sense and antisense strands. Regions of non-complementarity in the adapters can be used as primer binding sites to produce two distinct families of amplicons from the upper and lower DNA strands of each double-stranded fragment. In particular embodiments, non-identical primer binding sites can allow for the addition of pairs of non-identical sequencing adapters (e.g., P7 and P5 IIlumina adapters). Non-identical sequencing adapters can provide different landing sites for DNA sequencing primers that are used to sequence the DNA fragments in both directions. In particular embodiments, the length of the non-complementary region may include, for example, from 1 to 100 nucleotides, from 1 to 80 nucleotides, from 1 to 60 nucleotides, from 1 to 40 nucleotides, from 1 to 20 nucleotides, from 1 to 10 nucleotides, from 1 to 9 nucleotides, from 1 to 8 nucleotides, from 1 to 7 nucleotides, from 1 to 6 nucleotides, from 1 to 5 nucleotides, from 1 to 4 nucleotides, from 1 to 3 nucleotides, from 10 to 70 nucleotides, from 10 to 60 nucleotides, from 10 to 50 nucleotides, from 10 to 40 nucleotides, from 10 to 30 nucleotides, or from 10 to 20 nucleotides. In particular embodiments, the non-complementary region includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. The doubled-stranded portion of an asymmetrical adapter can include, for example, from 5 to 100 base pairs (bp), from 5 to 90 bp, from 5 to 80 bp, from 5 to 70 bp, from 5 to 60 bp, from 5 to 50 bp, from 5 to 40 bp, from 5 to 30 bp, from 5 to 20 bp, from 5 to 15 bp, or from 5 to 10 bp. In particular embodiments, the complementary region capable of forming a duplex structure includes 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 bp, or more, wherein the nucleotide sequence on the sense strand is complementary to the nucleotide sequence on the antisense strand.

In particular embodiments, an asymmetrical adapter is part of a nucleic acid that includes a unique barcode and a transposon end. In particular embodiments, the transposon end is double-stranded and the asymmetrical adapter that is part of the nucleic acid includes a single stranded region that forms a single stranded bubble. In particular embodiments, the unique barcode is double-stranded and the asymmetrical adapter that is part of the nucleic acid includes a double-stranded region of non-complementarity.

In particular embodiments, the asymmetrical adapters are forked adapters (also known as Y-shaped adapters). Forked adapters include a double-stranded region that can be annealed to a DNA fragment, and a flanking region of non-complementary, single-stranded nucleotides on the top and bottom strands.

In particular embodiments, the asymmetrical adapters are bubble adapters. A bubble adapter can refer to a DNA strand that contains a non-complementary, single stranded region between two complementary, double-stranded regions.

In particular embodiments, the asymmetrical adapters contain A-tails to facilitate binding to T-tailed, barcoded DNA fragments. In particular embodiments, the asymmetrical adapters contain T-tails to facilitate binding to A-tailed, barcoded DNA fragments.

Asymmetrical adapters are described in, for example, US20070172839, WO2009133466, CN102061335B, U.S. Pat. Nos. 8,420,319, 8,883,990, and Ahn et al. (2017) Scientific Reports 7:46678. Exemplary asymmetric adapter sequences can include an Illumina TruSeq universal adapter sequence 5′-AATGATACGGCGACCACCGAGATCTACACTCTTTCCCTACACGACGCTCTTCC GATCT-3′ (SEQ ID NO: 13) and an Illumina TruSeq Index adapter sequence 5′-GATCGGAAGAGCACACGTCTGAACTCCAGTCACNNNNNNATCTCGTATGCCGTCTTCT GCTTG-3′ (SEQ ID NO: 14), where “N” is any nucleotide, and the 6 Ns together are a unique sequence which can readily be identified as unique to a given sequencing library (Illumina, San Diego, Calif.). In particular embodiments, when the two single-stranded adapter sequences are annealed, there is a 12-nucleotide region of complementarity with the remaining nucleotides being non-complementary.

In particular embodiments, ligases are used to ligate asymmetrical adapters onto barcoded, fragmented DNA. In particular embodiments, a ligase is an enzyme that catalyzes intra- and intermolecular formation of phosphodiester bonds between 5′-phosphate and 3′-hydroxyl termini of nucleic acid strands. Ligases can include template-dependent or homologous ligases that seal nicks in double-stranded DNA. In particular embodiments, ligases can include NAD-type DNA ligases such as E. coli DNA ligase (available from e.g., New England BioLabs, Ipswich, Mass.), Tth DNA ligase (available from e.g., Thermo Fisher Scientific, Waltham, Mass.), AMPLIGASE® DNA ligase (Epicentre Technologies, Madison, Wis.), and ATP-type DNA ligases, such as T4 DNA ligase (available from e.g., New England BioLabs, Ipswich, Mass.) or FASTLINKT™ DNA ligase (Epicentre Technologies, Madison, Wis.).

(vi) Transposase-Based Systems. In particular embodiments, a transposase-based high accuracy system can include a plurality of transposases, each including a unique transposable barcode. In an in vitro transposition reaction described herein, the attachment of transposable barcodes to either end of a fragmented DNA leaves small gaps in between the 3′ ends of the fragmented DNA and the 5′ end of the non-transferred transposon ends, as depicted by arrows with large arrowheads in FIG. 1. These gaps need to be filled in by a DNA polymerase, an enzyme that can synthesize DNA polymers. In particular embodiments, the DNA polymerase uses the complementary strand as template to incorporate appropriate nucleotides during the synthesis. In particular embodiments, the DNA polymerase is template-independent. In particular embodiments, a DNA polymerase that lacks 5′-to-3′ exonuclease activity is used for to fill a gap. In particular embodiments where a nucleic acid including a double-stranded transposon end and a single-stranded unique barcode is present in a transposase-based system, as depicted in FIG. 1, the non-transferred strand needs to be displaced by a polymerase filling in the gaps, so that the polymerase can continue to synthesize DNA to the end of the DNA fragment to make the DNA end completely double-stranded. In particular embodiments, the DNA polymerase is a strand displacement/nick repair DNA polymerase. Examples of strand displacement/nick repair DNA polymerases that can be used in the systems and methods of the present disclosure include RepliPHI™ phi29 DNA polymerase (available from e.g., New England BioLabs, Ipswich, Mass.), DisplaceAce™ DNA polymerase (available from e.g., Epicentre Technologies, Madison, Wis.), rGka DNA polymerase (available from e.g., Epicentre Technologies, Madison, Wis.), SequiTherm™ DNA polymerase (available from e.g., Epicentre Technologies, Madison, Wis.), Taq DNA polymerase (available from e.g., New England BioLabs, Ipswich, Mass.), Tfl DNA polymerase (available from e.g., EURx, Gdansk, Poland), and MMLV reverse transcriptase (available from e.g., Promega, Madison, Wis.). In particular embodiments, a DNA polymerase of the present disclosure can fill in gaps created in an in vitro transposition reaction, displace non-transferred transposon ends, add A-tails, and/or add T-tails. In particular embodiments, a ligase as described above can join a 3′ end and a 5′ end of two strands of DNA after a gap has been filled by a polymerase. In particular embodiments of the systems and methods disclosed herein, a DNA polymerase allows the generation of barcoded, fragmented DNA molecules that are double-stranded, do not contain nicks or gaps, and have single-stranded A-tails or T-tails at the ends of the double-stranded fragmented DNA molecules ready for ligation to asymmetrical adapters. Therefore, in particular embodiments, the systems can include one or more of: (i) enzymes for nick repair/strand displacement, and (ii) enzymes for ligation of asymmetrical adapters.

In particular embodiments, a transposase-based system can be used to fragment and barcode target DNA. Target DNA can refer to any double-stranded DNA (dsDNA) of interest that is subjected to transposition with a transposase-based system described herein to generate barcoded DNA fragments. Target DNA can be derived from any in vivo or in vitro source, including from one or multiple cells, tissues, organs, or organisms, whether living or dead, or from any biological or environmental source (e.g., water, air, soil). In particular embodiments, target DNA includes eukaryotic and/or prokaryotic dsDNA that is derived from humans, animals, plants, fungi, bacteria, viruses, viroids, mycoplasma, or other microorganisms. In particular embodiments, target DNA includes genomic DNA, subgenomic DNA, chromosomal DNA, mitochondrial DNA, chloroplast DNA, plasmid or other episomal-derived DNA (or recombinant DNA contained therein), or double-stranded cDNA made by reverse transcription of RNA using an RNA-dependent DNA polymerase or reverse transcriptase to generate first-strand cDNA and then extending a primer annealed to the first-strand cDNA to generate dsDNA. In particular embodiments, the target DNA includes dsDNA that is prepared from all or a portion of one or more double-stranded or single-stranded DNA or RNA molecules using any methods known in the art, including methods for: DNA or RNA amplification; molecular cloning of all or a portion of one or more nucleic acid molecules in a plasmid, fosmid, BAC or other vector that subsequently is replicated in a suitable host cell; or capture of one or more nucleic acid molecules by hybridization, such as by hybridization to DNA probes on an array or microarray.

In particular embodiments, a transposase-based system of the present disclosure can include buffers, salts, ions, beads, and/or stabilizers that allow transposases, transposable barcodes, polymerases, and/or ligases to function in fragmenting DNA, barcoding DNA, adding A- or T-tails to the fragmented and barcoded DNA, and adding asymmetrical adapters to the fragmented and barcoded DNA.

(vii) Methods of Preparing a Nucleic Acid Sample for High Accuracy Sequencing. In particular embodiments, transposase reaction conditions are described in Vaezeslami et al. (2007) Bacteriol. 189(20): 7436-7441. In particular embodiments, the reaction includes a stage of loading the transposase with nucleic acids at a pH range of 6-9, preferably pH 7-8, in a 20-200 mM buffer, for example Tris buffer, which includes salt, such as KCl, at 0.1-0.8 M, and 5-50% glycerol. In particular embodiments, the nucleic acids are provided at 5-300 mM. In particular embodiments, the nucleic acids are provided at 5-300 μM. In particular embodiments, transposase is provided at 0.2-20 mg/ml. At the next stage, transposase complexes can be mixed with target DNA in the presence of 1-100 mM, preferably 5-20 mM Mn²⁺ or Mg²⁺ ions. In particular embodiments, the concentration of target DNA can include 0.000001-200 μg/ml. In particular embodiments, the concentration of target DNA can include 0.5-200 μg/ml. In particular embodiments, the concentration of target DNA can include 10-100 μg/ml. In particular embodiments, the amount of target DNA can include 10 ng, 20 ng, 30 ng, 40 ng, 50 ng, or more. In particular embodiments, the amount of target DNA can include 30 ng. In particular embodiments, Mn²⁺ ions can be used instead of Mg²⁺ ions.

In particular embodiments a method for preparing samples for high-accuracy sequencing can include contacting DNA samples with transposases that include transposable barcodes to produced barcoded DNA fragments. In particular embodiments, the barcoded DNA fragments can be contacted with one or more enzymes that perform nick repair/strand displacement and A-tailing to produce A-tailed, barcoded DNA fragments. In particular embodiments, the A-tailed, barcoded DNA fragments can be contacted with a ligase and asymmetrical adapters to produce a barcoded DNA library for amplification and high-accuracy sequencing. In particular embodiments, barcoded DNA fragments including asymmetrical adapters at the ends of the DNA fragments are ready for NGS and have been generated in one or two steps. In particular embodiments, generation of barcoded DNA fragments including asymmetrical adapters ready for NGS can occur in less than 4 hours, in less than 2 hours, in less than 1 hour, in less than 45 minutes, in less than 30 minutes, in less than 15 minutes, or less. In particular embodiments, generation of barcoded DNA fragments including asymmetrical adapters ready for NGS can occur within 120 minutes, within 105 minutes, within 90 minutes, within 75 minutes, within 60 minutes, within 45 minutes, within 30 minutes, within 15 minutes, or less of contacting a DNA sample with a plurality of transposases, each including a nucleic acid including a unique barcode and a transposon end; a polymerase; asymmetrical adapters; and a ligase.

Following efficient sample preparation as disclosed herein, particular embodiments can utilize NGS for sequencing. In particular embodiments, DNA sequencing of the barcoded DNA can be performed with commercially available NGS platforms by the following steps. First, the barcoded DNA sequencing libraries may be generated by clonal amplification by PCR in vitro. Second, the DNA may be immobilized on a support. Third, the spatially segregated, amplified DNA templates may be sequenced simultaneously in a massively parallel fashion without the requirement for a physical separation step. Sequencing may be by synthesis, such that the DNA sequence is determined by the addition of nucleotides to the complementary strand with reversible chain-termination chemistry. Sequencing may alternatively be by ligation, using a DNA ligase to join a probe oligonucleotide, labeled according to the position that will be sequenced, to an anchor sequence. While these steps are followed in most NGS platforms, each utilizes a different strategy (see e.g., Anderson and Schrijver, 2010, Genes 1: 38-69). For example, single molecule platforms do not amplify the DNA before sequencing. Examples of NGS platforms include:

| | | | Average |
|---|---|---|---|
| | | | Read |
| | Template | | Length |
| Platform | Preparation | Chemistry | (bases) |
| Roche 454 FLX ™ | Clonal-emulsion | Pyrosequencing | 400 |
| | PCR | | |
| Roche 454 | Clonal-emulsion | Pyrosequencing | 400 |
| TITANIUM ™ | PCR | | |
| Thermo Fisher | Clonal-emulsion | Proton generation | 200 |
| Scientific (Ion | PCR | with base | |
| Torrent) Ion systems | | incorporation | |
| Illumina MiSeq | Clonal Bridge | Reversible Dye | 35-300 |
| series | Amplification | Terminator | |
| Illumina MiniSeq | Clonal Bridge | Reversible Dye | 35-150 |
| series | Amplification | Terminator | |
| Illumina NextSeq | Clonal Bridge | Reversible Dye | 35-150 |
| series | Amplification | Terminator | |
| Thermo Fisher | Clonal-emulsion | Oligonucleotide | 35-50 |
| Scientific SOLiD ™ | PCR | Probe Ligation | |
| Complete Genomics | Rolling circle | Oligonucleotide | 62-120 |
| CGA | amplification | Probe Ligation | |
| Helicos Biosciences | Single Molecule | Reversible Dye | 35 |
| HELISCOPE ™ | | Terminator | |
| Pacific Biosciences | Single Molecule | Phospholinked | 10,000 |
| SMRT | | Fluorescent | |
| | | Nucleotides | |
| Oxford Nanopore | Single Molecule | Electrical signal | 10,000 |
| Technologies | | generated by base | |
| | | moving through | |
| | | nanopore | |

In particular embodiments, DNA segments can be enriched for target sequences of interest prior to NGS. In particular embodiments, to ensure adequate read depth, target sequences are enriched within the heterogeneous input sample to limit off-target sequence reads. Any known method of enrichment may be performed. In particular embodiments, the enrichment process is affinity purification, which relies on hybridization probes to preferentially bind target sequences of interest, for example in whole exome sequencing approaches. Mertes et al. (2011) Brief. Funct. Genomics 10: 374-386. In particular embodiments, the enrichment process is PCR amplification to increase the amount of target sequences of interest. Kinde et al. (2011) Sci. Transl. Med. 5: 167ra164. In embodiments where an amplification process is used to create a target-increased sample, this amplification would be a second amplification step. The second amplification can provide a stronger signal than if the second amplification was not performed.

(viii) Error Correction. An example of using sequence information from double stranded barcodes for error correction can be found in Schmitt et al. PNAS 109(36):14508-14513, US 2015/0024950, WO 2016/161177, and U.S. Pat. No. 9,752,188. In particular embodiments, adding double stranded barcodes to target DNA fragments can facilitate identification of library preparation and sequencing errors that can be removed computationally. The double stranded barcode labels/tags both strands of a fragmented nucleic acid molecule, allowing for utilization of family consensus information from both strands to computationally eliminate library preparation and sequencing errors and correct for DNA damage sites. For example, each strand of each copy of a double-stranded fragmented nucleic acid molecule, or portion thereof, produced by PCR amplification can be identified by its unique 5′ or 3′ barcode in combination with the use of asymmetrical adapters for strand discrimination. Individual sequence reads containing the same barcode are grouped into read families, and these sequence reads may be aligned. Consensus sequences may be derived from alignments of sequence reads in a given read family. In particular embodiments, a read family refers to sequence reads containing the same barcode and originating from the same nucleic acid molecule. In particular embodiments, a consensus sequence when used in reference to a read family refers to a common sequence derived from the reads in a family. In particular embodiments, a read family has at least three members before a consensus sequence is determined. Since mutation introduced by PCR error will not likely be found in PCR products from both strands at the same positions, a true mutation in a target nucleic acid molecule is likely to be present in both strands at the same position of nearly all or all of the copies present, which may be identified by their unique barcodes in combination with asymmetrical adapters for strand discrimination. In particular embodiments, a mutation in a target nucleic acid molecule is “called” (considered real and not an artifact) if it is observed in two or more read families.

In particular embodiments, processing of raw sequence reads involve the following: Initial processing of raw sequence reads can include family barcode trimming, adapter trimming and quality filtering. First, a family identifier for each read pair can be saved, including the barcode and transposon end sequences plus the first 13 nucleotides (nt) of the insert sequence from each read pair. Reads with Ns anywhere in this family identifier sequence can be discarded. The barcode and transposon end sequences can then be removed. In order to recognize the adaptor sequence on the 3′ end of the read for adapter trimming a minimum overlap of 10 nt at a maximum mismatch rate of 0.05 (i.e. 4 mismatches in 80 nt) can be required. Trimmed reads <50 nt can be discarded. Trimmed reads and quality scores can be exported into new FASTQ files which can be aligned using BWA to a full reference genome. Following alignment, paired reads can be further filtered based on the following criteria: (i) all reads can be required to be paired; (ii) if a target locus is specified, both reads in a pair can be required to overlap the target locus; (iii) each read in a pair can be required to have a minimum aligned sequence length of 50 nt; (iv) no Ns can be allowed in either pair; (v) nucleotide positions with a quality score<30 can be recorded as missing data; (vi) no more than 20% of the sequence in either pair is allowed to have a quality score lower than 30, or the entire read pair can be discarded; and finally, (vii) reads aligning to genomic regions containing low complexity or short-period tandem repeats, as identified by the repeat masking program ‘tantan’, can be discarded. Reads can then be ‘expanded’ by overlaying the read sequence on the reference using the CIGAR string, allowing family members to align properly in a consensus matrix. Read pairs can next be re-associated with their family IDs and sorted into their respective families. Families with fewer than 10 read-pair members can be discarded.

In particular embodiments, computational analysis to correct errors in sequencing can be performed on each read family as follows. A consensus matrix of the family can be made, and the consensus sequence taken at the 90% level. Positions with <90% consensus can be recorded as missing data. Read positions with a family read depth<10 can also be encoded as missing data (i.e. if a family consisted of 20 reads [10 read pairs] and 11 reads had missing data at position 5, the family consensus for position 5 is set to missing). Finally, the global site-specific mutational frequency is calculated by considering a consensus matrix of all family consensus sequences.

NGS performed without adding double stranded barcodes prior to library amplification can often have an error rate of 1%, or 1×10⁻² (1 error in 100 nucleotides). Thus, systems and methods of the present disclosure can be used in conjunction with NGS to yield an error rate that is lower than the error rate of NGS performed without the systems and methods described herein. In particular embodiments, high-accuracy sequencing can yield an error rate of 0.1%, 0.01%, 0.001%, 0.0001%, or 0.00001%. In particular embodiments, high-accuracy sequencing can yield an error rate of 1×10⁻³, 1×10⁻⁴, 1×10⁻⁵, 1×10⁻⁶, 1×10⁻⁷, 1×10⁻⁸, 1×10⁻⁹, 1×10⁻¹⁰, or 1×10⁻¹¹. In particular embodiments, high-accuracy sequencing can yield an error rate of 1×10⁻³, 2×10⁻³, 3×10⁻³, 4×10⁻³, 5×10⁻³, 6×10⁻³, 7×10⁻³, 8×10⁻³, or 9×10⁻³. In particular embodiments, high-accuracy sequencing can yield an error rate of 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴. In particular embodiments, high-accuracy sequencing can yield an error rate of 1×10⁻⁵, 2×10⁻⁵, 3×10⁻⁵, 4×10⁻⁵, 5×10⁻⁵, 6×10⁻⁵, 7×10⁻⁵, 8×10⁻⁵, or 9×10⁻⁵. In particular embodiments, high-accuracy sequencing can yield an error rate of 1×10⁻⁶, 2×10⁻⁶, 3×10⁻⁶, 4×10⁻⁶, 5×10⁻⁶, 6×10⁻⁶, 7×10⁻⁶, 8×10⁻⁶, or 9×10⁻⁶. In particular embodiments, high-accuracy sequencing can yield an error rate of 1×10⁻⁷, 2×10⁻⁷, 3×10⁻⁷, 4×10⁻⁷, 5×10⁻⁷, 6×10⁻⁷, 7×10⁻⁷, 8×10⁻⁷, or 9×10⁻⁷. In particular embodiments, high-accuracy sequencing can yield an error rate of 1×10⁻⁸, 2×10⁻⁸, 3×10⁻⁸, 4×10⁻⁸, 5×10⁻⁸, 6×10⁻⁸, 7×10⁻⁸, 8×10⁻⁸, or 9×10⁻⁸. In particular embodiments, high-accuracy sequencing can yield an error rate of 1×10⁻⁹, 2×10⁻⁹, 3×10⁻⁹, 4×10⁻⁹, 5×10⁻⁹, 6×10⁻⁹, 7×10⁻⁹, 8×10⁻⁹, or 9×10⁻⁹. In particular embodiments, high-accuracy sequencing can yield an error rate of 1×10⁻¹⁰, 2×10⁻¹⁰, 3×10⁻¹⁰, 4×10⁻¹⁰, 5×10⁻¹⁰, 6×10⁻¹⁰, 7×10⁻¹⁰, 8×10⁻¹⁰, or 9×10⁻¹⁰. In particular embodiments, high-accuracy sequencing can yield an error rate of 1×10⁻¹¹, 2×10⁻¹¹, 3×10⁻¹¹, 4×10⁻¹¹, 5×10⁻¹¹, 6×10⁻¹¹, 7×10⁻¹¹, 8×10⁻¹¹, or 9×10⁻¹¹. In particular embodiments, high-accuracy sequencing can yield an error rate of 1 error in 1000 nucleotides, 1 error in 10,000 nucleotides, 1 error in 100,000 nucleotides, 1 error in 1,000,000 nucleotides, 1 error in 10,000,000 nucleotides, 1 error in 100,000,000 nucleotides, 1 error in 1,000,000,000 nucleotides, 1 error in 10,000,000,000 nucleotides, or 1 error in 100,000,000,000 nucleotides. In particular embodiments, high-accuracy sequencing can yield an error rate of 9 errors in 1000 nucleotides, 8 errors in 1000 nucleotides, 7 errors in 1000 nucleotides, 6 errors in 1000 nucleotides, 5 errors in 1000 nucleotides, 4 errors in 1000 nucleotides, 3 errors in 1000 nucleotides, 2 errors in 1000 nucleotides, or 1 error in 1000 nucleotides. In particular embodiments, high-accuracy sequencing can yield an error rate of 9 errors in 10,000 nucleotides, 8 errors in 10,000 nucleotides, 7 errors in 10,000 nucleotides, 6 errors in 10,000 nucleotides, 5 errors in 10,000 nucleotides, 4 errors in 10,000 nucleotides, 3 errors in 10,000 nucleotides, 2 errors in 10,000 nucleotides, or 1 error in 10,000 nucleotides. In particular embodiments, high-accuracy sequencing can yield an error rate of 9 errors in 100,000 nucleotides, 8 errors in 100,000 nucleotides, 7 errors in 100,000 nucleotides, 6 errors in 100,000 nucleotides, 5 errors in 100,000 nucleotides, 4 errors in 100,000 nucleotides, 3 errors in 100,000 nucleotides, 2 errors in 100,000 nucleotides, or 1 error in 100,000 nucleotides. In particular embodiments, high-accuracy sequencing can yield an error rate of 9 errors in 1,000,000 nucleotides, 8 errors in 1,000,000 nucleotides, 7 errors in 1,000,000 nucleotides, 6 errors in 1,000,000 nucleotides, 5 errors in 1,000,000 nucleotides, 4 errors in 1,000,000 nucleotides, 3 errors in 1,000,000 nucleotides, 2 errors in 1,000,000 nucleotides, or 1 error in 1,000,000 nucleotides. In particular embodiments, high-accuracy sequencing can yield an error rate of 9 errors in 10,000,000 nucleotides, 8 errors in 10,000,000 nucleotides, 7 errors in 10,000,000 nucleotides, 6 errors in 10,000,000 nucleotides, 5 errors in 10,000,000 nucleotides, 4 errors in 10,000,000 nucleotides, 3 errors in 10,000,000 nucleotides, 2 errors in 10,000,000 nucleotides, or 1 error in 10,000,000 nucleotides. In particular embodiments, high-accuracy sequencing can yield an error rate of 9 errors in 100,000,000 nucleotides, 8 errors in 100,000,000 nucleotides, 7 errors in 100,000,000 nucleotides, 6 errors in 100,000,000 nucleotides, 5 errors in 100,000,000 nucleotides, 4 errors in 100,000,000 nucleotides, 3 errors in 100,000,000 nucleotides, 2 errors in 100,000,000 nucleotides, or 1 error in 100,000,000 nucleotides. In particular embodiments, high-accuracy sequencing can yield an error rate of 9 errors in 1,000,000,000 nucleotides, 8 errors in 1,000,000,000 nucleotides, 7 errors in 1,000,000,000 nucleotides, 6 errors in 1,000,000,000 nucleotides, 5 errors in 1,000,000,000 nucleotides, 4 errors in 1,000,000,000 nucleotides, 3 errors in 1,000,000,000 nucleotides, 2 errors in 1,000,000,000 nucleotides, or 1 error in 1,000,000,000 nucleotides. In particular embodiments, high-accuracy sequencing can yield an error rate of 9 errors in 10,000,000,000 nucleotides, 8 errors in 10,000,000,000 nucleotides, 7 errors in 10,000,000,000 nucleotides, 6 errors in 10,000,000,000 nucleotides, 5 errors in 10,000,000,000 nucleotides, 4 errors in 10,000,000,000 nucleotides, 3 errors in 10,000,000,000 nucleotides, 2 errors in 10,000,000,000 nucleotides, or 1 error in 10,000,000,000 nucleotides. In particular embodiments, high-accuracy sequencing can yield an error rate of 9 errors in 100,000,000,000 nucleotides, 8 errors in 100,000,000,000 nucleotides, 7 errors in 100,000,000,000 nucleotides, 6 errors in 100,000,000,000 nucleotides, 5 errors in 100,000,000,000 nucleotides, 4 errors in 100,000,000,000 nucleotides, 3 errors in 100,000,000,000 nucleotides, 2 errors in 100,000,000,000 nucleotides, or 1 error in 100,000,000,000 nucleotides.

(ix) Kits. Also disclosed herein are kits including one or more containers including one or more of components of the transposase-based systems described herein. In particular embodiments, components can be included which are useful for fragmenting DNA and/or useful for preparation of fragmented DNA for sequencing. The components of the kits can be provided in, or bound to, one or more solid materials. For example, one or more components can be provided in a container, which can be fabricated from plastic materials and formed in the shape of microfuge tubes or sequencing plates (e.g., 84- or 96-wells per plate). In particular embodiments, one or more components can be provided bound to a solid support. For example, one or more transposases can be bound via a tagging system as described above to a solid support such as beads or nanoparticles. The solid support can in turn be attached to the surface of a nylon membrane or to wells of a multi-well plate.

In particular embodiments, a kit can include one or more transposases of the disclosure. The transposase can be provided as a liquid solution (e.g., an aqueous or alcohol solution) in one or more containers. In particular embodiments, the transposase can be provided as a dried composition in one or more containers. In particular embodiments, each transposase is associated by non-covalent chemical bonding with a transposable barcode. In particular embodiments, two or more different transposases are provided in a single container or in two or more containers. Where two or more containers are provided, each container can include a single transposase, or one, some, or all of the containers can include a mixture of one, some, or all of the transposases. As noted above, two or more different transposase complexes having different recognition sequences can be used to reduce GC vs. AT bias and thus to provide superior control of fragmentation of genomic DNA. In particular embodiments, where two or more different transposase complexes are provided, the ratios of transposase complexes can be varied prior to packaging of the complexes in the kit. In particular embodiments, different ratios are suitable for different DNA targets and different kits can be manufactured for different types of targets.

In particular embodiments, a kit can include one or more transposable barcodes provided in one or more containers separate from transposases. In particular embodiments, the one or more transposable barcodes can be provided as a high diversity barcode library including more than 100,000, more than 125,000, more than 150,000, more than 175,000, more than 200,000, more than 225,000, more than 250,000, more than 275,000, more than 300,000, more than 325,000, more than 350,000, more than 375,000, more than 400,000, more than 425,000, more than 450,000, more than 475,000, more than 500,000, more than 525,000, more than 550,000, more than 575,000, more than 600,000, more than 625,000, more than 650,000, more than 675,000, more than 700,000, more than 725,000, more than 750,000, more than 775,000, more than 1,000,000 unique barcodes, or more. The transposable barcodes can be provided as a liquid solution (e.g., an aqueous or alcohol solution) in one or more containers. Alternatively, the transposable barcodes can be provided as a dried composition in one or more containers. In particular embodiments, two or more different transposable barcodes are provided in a single container or in two or more containers. Where two or more containers are provided, each container can include a single transposable barcode, or one, some, or all of the containers can include a mixture of one, some, or all of the transposable barcodes.

In particular embodiments, a kit can further include: a polymerase for strand displacement/nick repair of the DNA fragments; asymmetrical adapters; and a ligase. In particular embodiments, a kit can further include: control DNA for use in ensuring that the transposase complexes and other components of reactions are functioning properly (e.g., polymerases, ligases), buffers for enzymes, PCR reaction reagents (including buffers, dNTPs, amplification primers, PCR polymerases, fluorescent probes for quantitation and size estimation of DNA fragments), salts, detergents, activating cations (Mg²⁺ or Mn²⁺), beads for purification of DNA fragments, and wash solutions.

Optionally, the kits described herein include instructions for using the kit in the methods disclosed herein. In various embodiments, the kit may include instructions regarding preparation of components of the transposase-based sample/processing/error correction system; use of the components of the transposase-based system for preparation of DNA samples ready for sequencing in one or two steps that occur in less than 2 hours; instruction for interpreting results associated with using the kit (e.g., reference level of expected DNA yield, examples for interpreting high-accuracy sequencing results); proper disposal of the related waste; and the like. The instructions can be in the form of printed instructions provided within the kit or the instructions can be printed on a portion of the kit itself. Instructions may be in the form of a sheet, pamphlet, brochure, CD-Rom, or computer-readable device, or can provide directions to instructions at a remote location, such as a website. In particular embodiments, instruction for troubleshooting undesired experimental outcomes can be included.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A transposase including a nucleic acid including a barcode and a transposon end.
2. A transposase of embodiment 1, wherein the transposon end includes SEQ ID NOs: 4, 5, 6, 7, 8, 9, and/or 10.
3. A transposase of embodiment 1 or 2, wherein the transposon end is a mosaic end.
4. A nucleic acid of any of embodiments 1-3 further including a spacer sequence.
5. A transposase of any of embodiments 1-4 wherein the transposase includes a Tn3 transposase, a Tn5 transposase, a Tn7 transposase, a Tn10 transposase, a bacteriophage transposase, and/or a retroviral transposase.
6. A transposase of any of embodiments 1-5 wherein the transposase includes a E54K/L372P Tn5 transposase.
7. A transposase of any of embodiments 1-6 wherein the transposase includes SEQ ID NO: 1.
8. A transposase of any of embodiments 1-7 wherein the nucleic acid is selected from SEQ ID NOs: 2 and/or 11.
9. A transposase of any of embodiments 1-8 wherein the mosaic end includes SEQ ID NO: 4.
10. A transposase of any of embodiments 1-9 wherein the barcode is single-stranded.
11. A transposase of any of embodiments 1-9 wherein the barcode is double-stranded.
12. A transposase of any of embodiments 1-11 wherein the nucleic acid includes uracil and/or modified nucleotides.
13. A transposase of any of embodiments 1-12 wherein the nucleic acid includes a single stranded region that forms a single stranded bubble and a double-stranded transposon end.
14. A transposase of any of embodiments 1-12 wherein the nucleic acid includes a double-stranded region of non-complementarity, a double-stranded barcode, and a double-stranded transposon end.
15. A transposase of any of embodiments 1-12 wherein the nucleic acid includes an asymmetric adapter.
16. A transposase-based system for high-accuracy sequencing, including:

a plurality of transposases, each including a nucleic acid including a unique barcode and a transposon end,

a polymerase for nick repair/strand displacement;

asymmetrical adapters; and

a ligase.

17. A transposase-based system of embodiment 16 including at least 1,000; at least 10,000; at least 100,000; at least 1,000,000; at least 100,000,000; or at least 1,000,000,000 transposases.
18. A transposase-based system of embodiment 16 or 17, wherein at least one transposase includes a Tn3 transposase, a Tn5 transposase, a Tn7 transposase, a Tn10 transposase, a bacteriophage transposase, and/or a retroviral transposase.
19. A transposase-based system of any of embodiments 16-18, wherein at least one transposase includes E54K/L372P Tn5 transposase.
20. A transposase-based system of any of embodiments 16-19, wherein at least one transposase includes SEQ ID NO: 1.
21. A transposase-based system of any of embodiments 16-20, wherein at least one nucleic acid includes a single-stranded unique barcode and a double-stranded transposon end.
22. A transposase-based system of any of embodiments 16-20, wherein at least one nucleic acid includes a double-stranded unique barcode and a double-stranded transposon end.
23. A transposase-based system of any of embodiments 16-22, wherein at least one nucleic acid includes a unique barcode 5′ to the transposon end.
24. A transposase-based system of any of embodiments 16-23, wherein at least one nucleic acid is selected from SEQ ID NOs: 2 and 11.
25. A transposase-based system of any of embodiments 16-24, wherein the transposon end includes SEQ ID NOs: 4, 5, 6, 7, 8, 9, and/or 10.
26. A transposase-based system of any of embodiments 16-25, wherein the transposon end is a mosaic end.
27. A transposase-based system of any of embodiments 16-26, wherein the unique barcodes are based on Hamming codes.
28. A transposase-based system of any of embodiments 16-27, wherein at least one nucleic acid includes a single-stranded spacer.
29. A transposase-based system of any of embodiments 16-27, wherein at least one nucleic acid includes a double-stranded spacer.
30. A transposase-based system of any of embodiments 16-29, wherein the spacer is 5′ to the unique barcode.
31. A transposase-based system of any of embodiments 16-30, wherein the spacer includes a site for cleavage with a restriction enzyme.
32. A transposase-based system of any of embodiments 16-31 wherein the nucleic acid includes uracil and/or modified nucleotides.
33. A transposase-based system of any of embodiments 16-32 wherein the transposon end is double-stranded and each asymmetrical adapter is part of the nucleic acid and includes a single stranded region that forms a single stranded bubble.
34. A transposase-based system of any of embodiments 16-32 wherein the unique barcode is double-stranded and each asymmetrical adapter is part of the nucleic acid and includes a double-stranded region of non-complementarity.
35. A transposase-based system of any of embodiments 16-34 wherein the asymmetrical adapters are part of the nucleic acids.
36. A transposase-based system of any of embodiments 16-35, wherein the asymmetrical adapters include forked adapters.
37. A transposase-based system of any of embodiments 16-36, wherein the asymmetrical adapters include SEQ ID NOs: 13 and 14.
38. A transposase-based system of any of embodiments 16-37, wherein the asymmetrical adapters include 3′ T-overhangs.
39. A method for preparing a DNA sample for high-accuracy sequencing including:

Obtaining a DNA sample to be sequenced;

Contacting the DNA sample with:
a plurality of transposases, each including a nucleic acid including a unique barcode and a transposon end;
a polymerase for nick repair/strand displacement,
asymmetrical adapters, and
a ligase, thereby generating a DNA sample including barcoded, fragmented DNA including asymmetrical adapters; and

Amplifying by PCR the fragmented DNA, wherein the DNA sample including barcoded, fragmented DNA including asymmetrical adapters is ready for sequencing within 2 hours of the contacting step.

40. A method of embodiment 39, wherein the nucleic acid including a unique barcode and a transposon end is generated by annealing a barcoded transferred strand of the transposon end to its complementary non-transferred strand.
41. A method of embodiment 39 or 40, wherein the plurality of transposases are incubated with a plurality of nucleic acids, each including a unique barcode and a transposon end, for 30 minutes at room temperature before the contacting step.
42. A method of any of embodiments 39-41, wherein the contacting step is performed at 55° C. for 5 to 10 minutes.
43. A method of any of embodiments 39-42, wherein the polymerase removes non-transferred strand of the transposon end, fills in transferred strand complementary nucleotides, and/or adds an A-tail or a T-tail to the barcoded, fragmented DNA.
44. A method of any of embodiments 39-43, wherein the ligase attaches the asymmetrical adapters onto the ends of the barcoded, fragmented DNA.
45. A method of any of embodiments 39-44, wherein the barcoded, fragmented DNA including asymmetrical adapters is quantified and sized before the amplifying step by digital droplet PCR using primers including SEQ ID NOs: 15 and 16.
46. A method of any of embodiments 39-45, wherein contacting with a plurality of transposases occurs before contacting with asymmetrical adapters.
47. A method of any of embodiments 39-45, wherein contacting with a plurality of transposases occurs simultaneously with contacting with asymmetrical adapters.
48. A method of any of embodiments 39-47 including at least 1,000; at least 10,000; at least 100,000; at least 1,000,000; at least 100,000,000; or at least 1,000,000,000 transposases.
49. A method of any of embodiments 39-48, wherein at least one transposase includes a Tn3 transposase, a Tn5 transposase, a Tn7 transposase, a Tn10 transposase, a bacteriophage transposase, and/or a retroviral transposase.
50. A method of any of embodiments 39-49, wherein at least one transposase includes E54K/L372P Tn5 transposase.
51. A method of any of embodiments 39-50, wherein at least one transposase includes SEQ ID NO: 1.
52. A method of any of embodiments 39-51, wherein at least one nucleic acid includes a single-stranded unique barcode and a double-stranded transposon end.
53. A method of any of embodiments 39-51, wherein at least one nucleic acid includes a double-stranded unique barcode and a double-stranded transposon end.
54. A method of any of embodiments 39-53, wherein at least one nucleic acid includes a unique barcode 5′ to the transposon end.
55. A method of any of embodiments 39-54, wherein at least one nucleic acid is selected from SEQ ID NOs: 2 and 11.
56. A method of any of embodiments 39-55, wherein the transposon end includes SEQ ID NOs: 4, 5, 6, 7, 8, 9, and/or 10.
57. A method of any of embodiments 39-56, wherein the transposon end is a mosaic end.
58. A method of any of embodiments 39-57, wherein the unique barcodes are based on Hamming codes.
59. A method of any of embodiments 39-58, wherein at least one nucleic acid includes a single-stranded spacer.
60. A method of any of embodiments 39-58, wherein at least one nucleic acid includes a double-stranded spacer.
61. A method of any of embodiments 39-59, wherein the spacer is 5′ to the unique barcode.
62. A method of any of embodiments 39-61, wherein the spacer includes a site for cleavage with a restriction enzyme.
63. A method of any of embodiments 39-62, wherein the asymmetrical adapters provide non-identical primer binding sites for amplification of distinct PCR products derived from each complementary strand.
64. A method of any of embodiments 39-63 wherein the nucleic acid includes uracil and/or modified nucleotides.
65. A method of any of embodiments 39-64 wherein the transposon end is double-stranded and each asymmetric adapter is part of the nucleic acid and includes a single stranded region that forms a single stranded bubble.
66. A method of any of embodiments 39-64 wherein the unique barcode is double-stranded and each asymmetric adapter is part of the nucleic acid and includes a double-stranded region of non-complementarity.
67. A method of any of embodiments 39-66 wherein the asymmetric adapters are part of the nucleic acids.
68. A method of any of embodiments 39-67, wherein the asymmetrical adapters include forked adapters.
69. A method of any of embodiments 39-68, wherein the asymmetrical adapters include SEQ ID NOs: 13 and 14.
70. A method of any of embodiments 39-69, wherein the asymmetrical adapters include 3′ T-overhangs.
71. A method of any of embodiments 39-70, wherein the fragmented DNA sample includes 100-3000 bp in length.
72. A method of any of embodiments 39-71, wherein the DNA sample to be sequenced includes 10 ng to 50 ng.
73. A method of any of embodiments 39-72, wherein the amplifying step includes amplifying with primers including sequences complementary to each non-complementary region of each asymmetrical adapter.
74. A method of any of embodiments 39-73, wherein the high accuracy sequencing yields an error rate of 1×10⁻⁶ to 1×10⁻¹¹.
75. A method including incubating DNA with transposases including high diversity barcodes to generate fragmented DNA including the high diversity barcodes.
76. A method of embodiment 75, wherein the DNA is genomic DNA.
77. A method of embodiment 75 or 76, wherein the high diversity barcodes are based on Hamming codes.
78. A method of any of embodiments 75-77 including computationally correcting errors introduced into the barcodes by a polymerase.
79. A method of any of embodiments 75-78 including ligating asymmetrical adapters to the fragmented DNA.
80. A method of any of embodiments 75-79 including quantifying and sizing the fragmented DNA by digital droplet PCR.
81. A method of any of embodiments 75-80 including amplifying the fragmented DNA for sequencing.
82. A method of any of embodiments 75-81 including sequencing the DNA.
83. A method of any of embodiments 75-82 including eliminating sequence errors computationally via generation of a consensus sequence from collapse of sequence reads which arise from each same fragmented DNA molecule.
84. A kit including:

A plurality of transposases;

A plurality of nucleic acid molecules, each nucleic acid molecule including a transposon end and a unique barcode;

A polymerase;

Asymmetric adapters; and

A ligase.

85. A kit of embodiment 84, wherein at least one nucleic acid includes a single-stranded unique barcode and a double-stranded transposon end.
86. A kit of embodiment 84, wherein at least one nucleic acid includes a double-stranded unique barcode and a double-stranded transposon end.
87. A kit of any of embodiments 84-86, wherein at least one nucleic acid includes a unique barcode 5′ to the transposon end.
88. A kit of any of embodiments 84-87, wherein at least one nucleic acid is selected from SEQ ID NOs: 2 and 11.
89. A kit of any of embodiments 84-88, wherein at least one mosaic end includes SEQ ID NOs: 4, 5, 6, 7, 8, 9, and/or 10.
90. A kit of any of embodiments 84-89, wherein the transposon end is a mosaic end.
91. A kit of any of embodiments 84-90, wherein the unique barcodes are based on Hamming codes.
92. A kit of any of embodiments 84-91, wherein at least one nucleic acid includes a single-stranded spacer.
93. A kit of any of embodiments 84-92, wherein at least one nucleic acid includes a double-stranded spacer.
94. A kit of any of embodiments 84-93, wherein the spacer is 5′ to the unique barcode.
95. A kit of any of embodiments 84-94, wherein the spacer includes a site for cleavage with a restriction enzyme.
96. A kit of any of embodiments 84-95, wherein the nucleic acid molecules include a library of transposable high diversity barcodes.
97. A kit of any of embodiments 84-96, wherein the at least one transposase includes a Tn3 transposase, a Tn5 transposase, a Tn7 transposase, a Tn10 transposase, a bacteriophage transposase, and/or a retroviral transposase.
98. A kit of any of embodiments 84-97, wherein the at least one transposase includes E54K/L372P Tn5 transposase.
99. A kit of any of embodiments 84-98, wherein the at least one transposase includes SEQ ID NO: 1.
100. A kit of any of embodiments 84-99 wherein the nucleic acid molecule includes uracil and/or modified nucleotides.
101. A kit of any of embodiments 84-100 wherein the transposon end is double-stranded and each asymmetrical adapter is part of the nucleic acid and includes a single stranded region that forms a single stranded bubble.
102. A kit of any of embodiments 84-100 wherein the unique barcode is double-stranded and each asymmetrical adapter is part of the nucleic acid and includes a double-stranded region of non-complementarity.
103. A kit of any of embodiments 84-102 wherein the asymmetrical adapters are part of the nucleic acids.
104. A kit of any of embodiments 84-103, wherein the asymmetrical adapters include forked adapters.
105. A kit of any of embodiments 84-104, wherein the asymmetrical adapters include SEQ ID NOs: 13 and 14.
106. A kit of any of embodiments 84-105, wherein the asymmetrical adapters include 3′ T-overhangs.
107. A kit of any of embodiments 84-106 further including primers including SEQ ID NOs: 15 and 16 for quantitation and/or sizing.
108. A kit of any of embodiments 84-107 further including buffers, dNTPs, and/or fluorescent probes.
109. A kit of any of embodiments 84-108 further including primers for sequencing.

Variants of nucleotide and protein sequences disclosed or described herein are also included. In particular embodiments, a protein can include one or more insertions, one or more deletions, one or more amino acid substitutions (e.g., conservative amino acid substitutions or non-conservative amino acid substitutions), or a combination of the above-noted changes, when compared with the disclosed or described proteins (e.g., SEQ ID NO: 1, FIG. 2). An insertion, deletion or substitution may be anywhere in a protein disclosed or described herein, including at the amino- or carboxy-terminus or both ends of this region, provided that the expression of the modified protein can still be used in an in vitro transposition reaction to fragment and barcode DNA. A “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (GLn); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

In particular embodiments, a nucleotide sequence of a nucleic acid disclosed or described herein can include one or more insertions, one or more deletions, one or more base substitutions, one or more base modifications. In particular embodiments, nucleotide modifications and/or nucleic acid modifications include uracil, 2-aminopurine, 2,6-diaminopurine, 5-bromo-deoxyuridine, deoxyuridine, inverted dT, inverted dideoxy-T, dideoxycytidine, 5-methyl deoxycytidine, deoxyinosine, 5-hydroxybutynl-2′-deoxyuridine, 8-aza-7-deazaguanosine, locked nucleic acids (LNA), peptide nucleic acid (PNA), 5-nitroindole, 2′-O-methyl RNA bases, hydroxymethyl deoxycytidine, isodeoxycytidine, isodeoxyguanine, fluoro bases, morpholino subunit, universal-binding nucleotide (such as C-phenyl, C-naphthyl, inosine, azole carboxamide, l-β-D-ribofuranosyl-4-nitroindole, 1-P-D-ribofuranosyl-5-nitroindole, 1-P-D-ribofuranosyl-6-nitroindole, L-β-D-ribofuranosyl-3-nitropyrrole), 2′-sugar substitution (such as a 2′-O-methyl, 2′-O-methoxy ethyl, 2′-O-2-methoxy ethyl, 2′-O-allyl, or halogen like 2′-fluoro), modified internucleotide linkages (such as phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, selenophosphate, thionophosphoramidate, thionoalkylphosphonate, thionoalkylphosphotriester, or boranophosphate linkage), or a combination of the above-noted changes, when compared with the disclosed or described nucleotide sequences (e.g., SEQ ID NOs: 2-16). An insertion, deletion, substitution, or modification may be anywhere in a nucleotide sequence disclosed or described herein, including at the 5′ end, 3′ end, or both ends, provided that the nucleic acid can still be used in the systems and methods described herein.

Variants of the protein or nucleic acid sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to a protein or nucleic acid sequence described or disclosed herein.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

### Example 1

Tn5 Transposase for Error Correction. Incubate Tn5 transposase loaded with high diversity barcodes (these can be double or single stranded) with genomic DNA. Insertion of DNA barcode and fragmentation occurs in a single 5-10 min step. The nicked strand is displaced during polymerization. A-tailing can occur in this step as well. Following nick repair/strand displacement and A-tailing, ligation of asymmetric forked adapters on the barcoded fragmented DNA is performed. This ligation step can occur via A/T mediated base pairing or can incorporate nucleotide overhangs created by cleavage of a restriction site embedded in a spacer region included in each transposable barcode. Either way, PCR using primers that anneal to the non-complementary regions of the forked adapters (not shown) amplify the library for sequencing. The forked adapters permit deconvolution of strand specific sequence. At this point the library can be sequenced directly or subjected to gene/region specific enrichment (not shown) prior to sequencing. Potential errors introduced in the barcode by taq polymerase can be corrected computationally. This is further simplified when generalized/known (but still high diversity) barcodes are designed based on Hamming codes. Errors introduced via library preparation, etc. can be eliminated computationally via the collapse of reads which arose from the same molecule (i.e., the error-corrected sequence is generated by filtering for sites with, for example >90% consensus within each barcode family).

### Example 2

Materials and Methods. Transposon Primers. PAGE-purified, 5′ phosphorylated transposable-element primers containing the hyperactive Mosaic End (ME) sequence (bold) and were obtained from IDT (Integrated DNA Technologies, Coralville, Iowa): Transferred strand: 5′-[phos]NNNNNNNNNNAGATGTGTATAAGAGACAG (SEQ ID NO: 11); Non-transferred strand: 5′-[phos]CTGTCTCTTATACA[ddC] (SEQ ID NO: 12).

Primers were combined at 10 μM each and annealed by incubation at 95° C. for 3 minutes, 70° C. for 3 minutes, and 70° C. to 26° C. decreasing 1° C. per 30-second cycle. Annealed primers were diluted 1:1 in 100% glycerol.

Transposome Formation. An equal volume of diluted primers and EZ-Tn5 (# TNP92110, Lucigen, Middleton, Wis.) were combined and allowed to bind for 30 minutes at room temperature.

Tagment DNA. Thirty nanograms of HCT116 DNA were combined with 2.5 μL of formed transposome and tagmented at 55° C. for 8 minutes. The tagmentation was terminated by the addition of Neutralize Tagment Buffer (Illumina, San Diego, Calif.). Tagmentation reactions were cleaned with 1.8 volumes of AMPure XP magnetic beads (# A63880, Beckman Coulter, Brea, Calif.).

Overhang Fill-in. Removal of non-transferred strand transposable-element primers and fill-in of transferred strand complementary nucleotides was achieved by addition of an equal volume of Phusion Master Mix (# F531S, Thermo Fisher Scientific, Waltham, Mass.) to cleaned, tagmented DNA with incubation at 60° C. for 5 minutes. Fill-in reactions were cleaned with 1.8 volumes of AMPure XP magnetic beads.

3′ Adenine Addition. Adenine bases were added to the 3′ termini of the tagmented DNA fragments by the addition of 200 μM final concentration dATP (# N04405, New England Biolabs, Ipswich, Mass.) and 2.5 U per reaction of Klenow (3′ to 5′ exo-, 5U/μL) (# M0212S, New England Biolabs, Ipswich, Mass.). Reactions were incubated at 37° C. for 30 minutes.

Adapter Ligation. Sequencing libraries were formed by ligation of TruSeq Adapter Indexes (Illumina) and tagmented DNA fragments using 0.2 U per reaction of T4 DNA Ligase (# M02025, New England Biolabs, Ipswich, Mass.). Sequencing libraries were cleaned with 1 volume of AMPure XP magnetic beads and quantified by ddPCR using Quantisize primers (Laurie et al. (2013) BioTechniques 55: 61-67):

Primer 1:
(SEQ ID NO: 15)
5′-AATGATACGGCGACCACCGA
Primer 2:
(SEQ ID NO: 16)
5′-CAAGCAGAAGACGGCATACGA

One million molecules of a sequencing library were amplified per reaction using Quantisize primers and TruSeq PCR Master Mix (Illumina, San Diego, Calif.), and thermal cycled at 98° C. for 30 seconds, then 15 cycles of: 98° C. for 10 seconds, 64° C. for 30 seconds, and 72° C. for 30 seconds; followed by 72° C. for 5 minutes.

Sequencing. Libraries were sequenced on a MiSeq instrument using 2×150 paired-end sequencing (# MS-102-2002, Illumina, San Diego, Calif.). Read mapping quality was Q30.

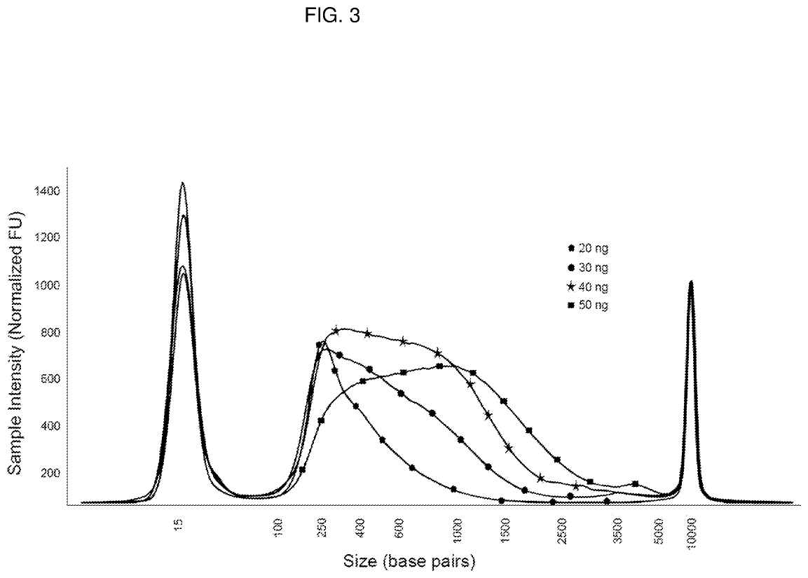

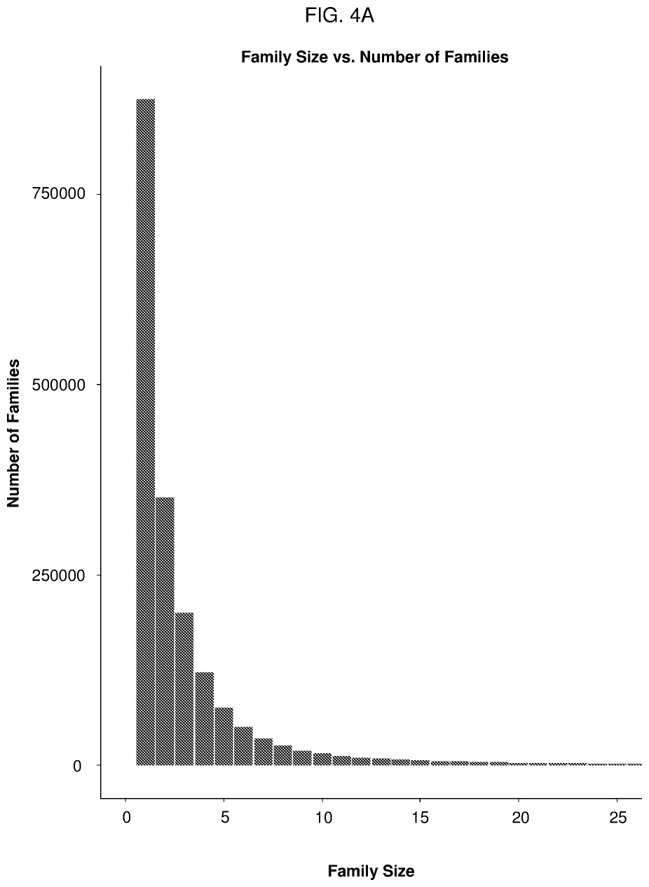

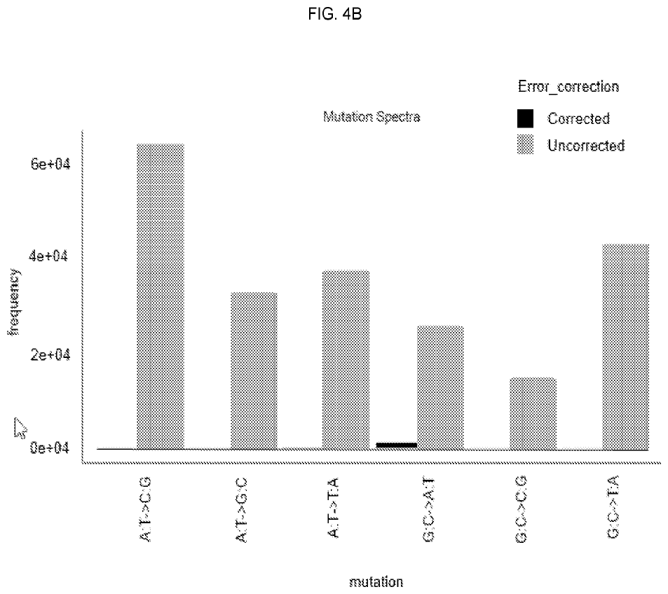

Barcoded DNA fragments of HCT116 genomic DNA were generated as described in the Materials and Methods. Analysis of the tagmented DNA showed that size distribution of tagmented genomic DNA fragments decreases with decreasing DNA input mass (FIG. 3). Following tagmentation and ligation of adapters, the DNA fragments were PCR amplified and sequenced. The distribution of barcode families in the sequencing library is shown in FIG. 4A. A large number (>750,000) of barcode families are associated with only 1 member (a family size of 1). However, >187,500 barcode families are associated with 3 or more members, which render these barcodes families useful for generation of consensus sequences and thus for error correction. FIG. 4B shows the frequency of indicated mutations of an error-corrected genomic sequence versus an uncorrected genomic sequence. The uncorrected genomic sequence has frequencies of >1 mutation in 10,000 nucleotides to >6 in 10,000 nucleotides. Error correction using the barcoded DNA fragments decreased the frequency of these mutations to zero or near zero.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in the ability to prepare a fragmented and barcoded DNA sample ready for NGS in less than 2 hours or to distinguish errors that occur during sample preparation for genetic sequencing from rare sequence variants.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A transposase comprising a nucleic acid comprising a barcode and a transposon end.

2. A transposase of claim 1, wherein the transposon end comprises SEQ ID NOs: 4, 5, 6, 7, 8, 9, and/or 10.

3. A transposase of claim 1, wherein the transposon end is a mosaic end.

4. A transposase of claim 3, wherein the mosaic end comprises SEQ ID NO: 4.

5. A nucleic acid of claim 1, further comprising a spacer sequence.

6. A nucleic acid of claim 1, wherein the nucleic acid molecule comprises uracil and/or modified nucleotides.

7. A nucleic acid of claim 1, further comprising a single stranded region that forms a single stranded bubble, and wherein the transposon end is double-stranded.

8. A nucleic acid of claim 1, further comprising a double-stranded region of non-complementarity, and wherein the unique barcode is double-stranded.

9. A nucleic acid of claim 1, further comprising an asymmetrical adapter.

10. A transposase of claim 1, wherein the transposase comprises a Tn3 transposase, a Tn5 transposase, a Tn7 transposase, a Tn10 transposase, a bacteriophage transposase, and/or a retroviral transposase.

11. A transposase of claim 1, wherein the transposase comprises a E54K/L372P Tn5 transposase.

12. A transposase of claim 1, wherein the transposase comprises SEQ ID NO: 1.

13. A transposase of claim 1, wherein the nucleic acid is selected from SEQ ID NOs: 2 and/or 11.

14. A transposase-based system for high-accuracy sequencing, comprising:
a plurality of transposases, each comprising a nucleic acid comprising a unique barcode and a transposon end,
a polymerase for strand displacement/nick repair;
asymmetrical adapters; and
a ligase.

15. A transposase-based system of claim 14, comprising at least 1,000; at least 10,000; at least 100,000; at least 1,000,000; at least 100,000,000; or at least 1,000,000,000 transposases.

16. A transposase-based system of claim 14, wherein at least one transposase comprises a Tn3 transposase, a Tn5 transposase, a Tn7 transposase, a Tn10 transposase, a bacteriophage transposase, and/or a retroviral transposase.

17. A transposase-based system of claim 14, wherein at least one transposase comprises E54K/L372P Tn5 transposase.

18. A transposase-based system of claim 14, wherein at least one transposase comprises SEQ ID NO: 1.

19. A transposase-based system of claim 14, wherein at least one nucleic acid comprises a single-stranded unique barcode and a double-stranded transposon end.

20. A transposase-based system of claim 14, wherein at least one nucleic acid comprises a double-stranded unique barcode and a double-stranded transposon end.

21. A transposase-based system of claim 14, wherein at least one nucleic acid comprises a unique barcode 5′ to the transposon end.

22. A transposase-based system of claim 14, wherein at least one nucleic acid is selected from SEQ ID NOs: 2 and 11.

23. A transposase-based system of claim 14, wherein the transposon end comprises SEQ ID NOs: 4, 5, 6, 7, 8, 9, and/or 10.

24. A transposase-based system of claim 14, wherein the transposon end is a mosaic end.

25. A transposase-based system of claim 14, wherein the unique barcodes are based on Hamming codes.

26. A transposase-based system of claim 14, wherein at least one nucleic acid comprises a single-stranded spacer.

27. A transposase-based system of claim 14, wherein at least one nucleic acid comprises a double-stranded spacer.

28. A transposase-based system of claim 14, wherein the spacer is 5′ to the unique barcode.

29. A transposase-based system of claim 14, wherein the spacer comprises a site for cleavage with a restriction enzyme.

30. A transposase-based system of claim 14, wherein the nucleic acid comprises uracil and/or modified nucleotides.

31. A transposase-based system of claim 14, wherein the transposon end is double-stranded and each asymmetrical adapter is part of the nucleic acid and comprises a single stranded region that forms a single stranded bubble.

32. A transposase-based system of claim 14, wherein the unique barcode is double-stranded and each asymmetrical adapter is part of the nucleic acid and comprises a double-stranded region of non-complementarity.

33. A transposase-based system of claim 14, wherein the asymmetrical adapters are part of the nucleic acids.

34. A transposase-based system of claim 14, wherein the asymmetrical adapters comprise forked adapters.

35. A transposase-based system of claim 14, wherein the asymmetrical adapters comprise SEQ ID NOs: 13 and 14.

36. A transposase-based system of claim 14, wherein the asymmetrical adapters comprise 3′ T-overhangs.

37. A method for preparing a DNA sample for high-accuracy sequencing comprising:
Obtaining a DNA sample to be sequenced;
Contacting the DNA sample with:
a plurality of transposases, each comprising a nucleic acid comprising a unique barcode and a transposon end;
a polymerase for strand displacement/nick repair,
asymmetrical adapters, and
a ligase, thereby generating a DNA sample comprising barcoded, fragmented DNA comprising asymmetrical adapters; and
Amplifying by PCR the fragmented DNA, wherein the DNA sample comprising barcoded, fragmented DNA comprising asymmetrical adapters is ready for sequencing within 2 hours of the contacting step.

38. A method of claim 37, wherein the nucleic acid comprising a unique barcode and a transposon end is generated by annealing a barcoded transferred strand of the transposon end to its complementary non-transferred strand.

39. A method of claim 37, wherein the plurality of transposases are incubated with a plurality of nucleic acids, each comprising a unique barcode and a transposon end, for 30 minutes at room temperature before the contacting step.

40. A method of claim 37, wherein the contacting step is performed at 55° C. for 5 to 10 minutes.

41. A method of claim 37, wherein the polymerase removes non-transferred strand of the transposon end, fills in transferred strand complementary nucleotides, and/or adds an A-tail or a T-tail to the barcoded, fragmented DNA.

42. A method of claim 37, wherein the ligase attaches the asymmetrical adapters onto the ends of the barcoded, fragmented DNA.

43. A method of claim 37, wherein the barcoded, fragmented DNA comprising asymmetrical adapters is quantified and sized before the amplifying step by digital droplet PCR using primers comprising SEQ ID NOs: 15 and 16.

44. A method of claim 37, wherein contacting with a plurality of transposases occurs before contacting with asymmetrical adapters.

45. A method of claim 37, wherein contacting with a plurality of transposases occurs simultaneously with contacting with asymmetrical adapters.

46. A method of claim 37 comprising at least 1,000; at least 10,000; at least 100,000; at least 1,000,000; at least 100,000,000; or at least 1,000,000,000 transposases.

47. A method of claim 37, wherein at least one transposase comprises a Tn3 transposase, a Tn5 transposase, a Tn7 transposase, a Tn10 transposase, a bacteriophage transposase, and/or a retroviral transposase.

48. A method of claim 37, wherein at least one transposase comprises E54K/L372P Tn5 transposase.

49. A method of claim 37, wherein at least one transposase comprises SEQ ID NO: 1.

50. A method of claim 37, wherein at least one nucleic acid comprises a single-stranded unique barcode and a double-stranded transposon end.

51. A method of claim 37, wherein at least one nucleic acid comprises a double-stranded unique barcode and a double-stranded transposon end.

52. A method of claim 37, wherein at least one nucleic acid comprises a unique barcode 5′ to the transposon end.

53. A method of claim 37, wherein at least one nucleic acid is selected from SEQ ID NOs: 2 and 11.

54. A method of claim 37, wherein the transposon end comprises SEQ ID NOs: 4, 5, 6, 7, 8, 9, and/or 10.

55. A method of claim 37, wherein the transposon end is a mosaic end.

56. A method of claim 37, wherein the unique barcodes are based on Hamming codes.

57. A method of claim 37, wherein at least one nucleic acid comprises a single-stranded spacer.

58. A method of claim 37, wherein at least one nucleic acid comprises a double-stranded spacer.

59. A method of claim 37, wherein the spacer is 5′ to the unique barcode.

60. A method of claim 37, wherein the spacer comprises a site for cleavage with a restriction enzyme.

61. A method of claim 37, wherein the asymmetrical adapters provide non-identical primer binding sites for amplification of distinct PCR products derived from each complementary strand.

62. A method of claim 37, wherein the nucleic acid comprises uracil and/or modified nucleotides.

63. A method of claim 37, wherein the transposon end is double-stranded and each asymmetrical adapter is part of the nucleic acid and comprises a single stranded region that forms a single stranded bubble.

64. A method of claim 37, wherein the unique barcode is double-stranded and each asymmetrical adapter is part of the nucleic acid and comprises a double-stranded region of non-complementarity.

65. A method of claim 37, wherein the asymmetrical adapters are part of the nucleic acids.

66. A method of claim 37, wherein the asymmetrical adapters comprise forked adapters.

67. A method of claim 37, wherein the asymmetrical adapters comprise SEQ ID NOs: 13 and 14.

68. A method of claim 37, wherein the asymmetrical adapters comprise 3′ T-overhangs.

69. A method of any of claim 37, wherein the fragmented DNA sample comprises 100-3000 bp in length.

70. A method of claim 37, wherein the DNA sample to be sequenced comprises 10 ng to 50 ng.

71. A method of claim 37, wherein the amplifying step comprises amplifying with primers comprising sequences complementary to each non-complementary region of each asymmetrical adapter.

72. A method of claim 37, wherein the high accuracy sequencing yields an error rate of 1×10⁻⁶ to 1×10⁻¹¹.

73. A method comprising incubating DNA with transposases comprising high diversity barcodes to generate fragmented DNA comprising the high diversity barcodes.

74. A method of claim 73, wherein the DNA is genomic DNA.

75. A method of claim 73, wherein the high diversity barcodes are based on Hamming codes.

76. A method of claim 73, comprising computationally correcting errors introduced into the barcodes by a polymerase.

77. A method of claim 73, comprising ligating asymmetrical adapters to the fragmented DNA.

78. A method of claim 73, comprising quantifying and sizing the fragmented DNA by digital droplet PCR.

79. A method of claim 73, comprising amplifying the fragmented DNA for sequencing.

80. A method of claim 73, comprising sequencing the DNA.

81. A method of claim 73, comprising eliminating sequence errors computationally via generation of a consensus sequence from collapse of sequence reads which arise from each same fragmented DNA molecule.

82. A kit comprising:
A plurality of transposases;
A plurality of nucleic acid molecules, each nucleic acid molecule comprising a transposon end and a unique barcode;
A polymerase;
Asymmetric adapters; and
A ligase.

83. A kit of claim 82, wherein at least one nucleic acid comprises a single-stranded unique barcode and a double-stranded transposon end.

84. A kit of claim 82, wherein at least one nucleic acid comprises a double-stranded unique barcode and a double-stranded transposon end.

85. A kit of claim 82, wherein at least one nucleic acid comprises a unique barcode 5′ to the transposon end.

86. A kit of claim 82, wherein at least one nucleic acid is selected from SEQ ID NOs: 2 and 11.

87. A kit of claim 82, wherein at least one mosaic end comprises SEQ ID NOs: 4, 5, 6, 7, 8, 9, and/or 10.

88. A kit of claim 82, wherein the transposon end is a mosaic end.

89. A kit of claim 82, wherein the unique barcodes are based on Hamming codes.

90. A kit of claim 82, wherein at least one nucleic acid comprises a single-stranded spacer.

91. A kit of claim 82, wherein at least one nucleic acid comprises a double-stranded spacer.

92. A kit of claim 82, wherein the spacer is 5′ to the unique barcode.

93. A kit of claim 82, wherein the spacer comprises a site for cleavage with a restriction enzyme.

94. A kit of claim 82, wherein the nucleic acid molecules comprise a library of transposable high diversity barcodes.

95. A kit of claim 82, wherein the at least one transposase comprises a Tn3 transposase, a Tn5 transposase, a Tn7 transposase, a Tn10 transposase, a bacteriophage transposase, and/or a retroviral transposase.

96. A kit of claim 82, wherein the at least one transposase comprises E54K/L372P Tn5 transposase.

97. A kit of claim 82, wherein the at least one transposase comprises SEQ ID NO: 1.

98. A kit of claim 82, wherein the nucleic acid molecule comprises uracil and/or modified nucleotides.

99. A kit of claim 82, wherein the transposon end is double-stranded and each asymmetrical adapter is part of the nucleic acid and comprises a single stranded region that forms a single stranded bubble.

100. A kit of claim 82, wherein the unique barcode is double-stranded and each asymmetrical adapter is part of the nucleic acid and comprises a double-stranded region of non-complementarity.

101. A kit of claim 82, wherein the asymmetrical adapters are part of the nucleic acids.

102. A kit of claim 82, wherein the asymmetrical adapters comprise forked adapters.

103. A kit of claim 82, wherein the asymmetrical adapters comprise SEQ ID NOs: 13 and 14.

104. A kit of claim 82, wherein the asymmetrical adapters comprise 3′ T-overhangs.

105. A kit of claim 82 further comprising primers comprising SEQ ID NOs: 15 and 16 for quantitation and/or sizing.

106. A kit of claim 82 further comprising buffers, dNTPs, and/or fluorescent probes.

107. A kit of claim 82 further comprising primers for sequencing.


SEQUENCE LISTING (16 sequences)

SEQ ID NO: 1 - protein, 450 aa, Artificial sequence
  E54K/L372P Tn5 transposase
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALW

SEQ ID NO: 2 - DNA, 31 bp, Artificial sequence
  exemplary transposable barcode; the 5′ end of the nucleic acid sequence has a phosphate group
  misc_feature (1)..(12): n can be any nucleotide; the 5′ end of the nucleic acid sequence has a phosphate group
nnnnnnnnnnnnctgtctcttatacacatct

SEQ ID NO: 3 - DNA, 19 bp, Escherichia coli
ctgactcttatacacaagt

SEQ ID NO: 4 - DNA, 19 bp, Artificial sequence
  mosaic end; artificial mutant of Tn5 outer end
ctgtctcttatacacatct

SEQ ID NO: 5 - DNA, 28 bp, Artificial sequence
  transposon end sequence derived from mariner/Tc1 Mos1 transposon
aaacgacatttcatacttgtacacctga

SEQ ID NO: 6 - DNA, 25 bp, Artificial sequence
  transposon end sequence derived from mariner/Tc1 Mos1 transposon
tttgctgtaaagtatgaacatgtgg

SEQ ID NO: 7 - DNA, 17 bp, Artificial sequence
  transposon end sequence derived from Musca domestica Hermes transposon
cttgttgttgttctctg

SEQ ID NO: 8 - DNA, 17 bp, Artificial sequence
  transposon end sequence derived from Musca domestica Hermes transposon
gaacaacaacaagagac

SEQ ID NO: 9 - DNA, 17 bp, Artificial sequence
  transposon end sequence derived from Musca domestica Hermes transposon
cttgttgaagttctctg

SEQ ID NO: 10 - DNA, 17 bp, Artificial sequence
  transposon end sequence derived from Musca domestica Hermes transposon
gaacaacttcaagagac

SEQ ID NO: 11 - DNA, 29 bp, Artificial sequence
  exemplary transposable barcode; the 5′ end of the nucleic acid sequence has a phosphate group
  misc_feature (1)..(10): n can be any nucleotide; the 5′ end of the nucleic acid sequence has a phosphate group
nnnnnnnnnnagatgtgtataagagacag

SEQ ID NO: 12 - DNA, 15 bp, Artificial sequence
  Non-transferred strand of transposable barcode described in Example 2; the 5′ end of the nucleic acid sequence has a phosphate group
  modified_base (15)..(15): c is dideoxycytidine; the 5′ end of the nucleic acid sequence has a phosphate group
ctgtctcttatacac

SEQ ID NO: 13 - DNA, 58 bp, Artificial sequence
  Illumina TruSeq universal adapter sequence
aatgatacggcgaccaccgagatctacactctttccctacacgacgctcttccgatct

SEQ ID NO: 14 - DNA, 63 bp, Artificial sequence
  Illumina TruSeq Index adapter sequence
  misc_feature (34)..(39): n can be any nucleotide
gatcggaagagcacacgtctgaactccagtcacnnnnnnatctcgtatgccgtcttctgcttg

SEQ ID NO: 15 - DNA, 20 bp, Artificial sequence
  forward Quantisize primer
aatgatacggcgaccaccga

SEQ ID NO: 16 - DNA, 21 bp, Artificial sequence
  reverse Quantisize primer
caagcagaagacggcatacga